SEQUENCE LISTING (5 sequences)

SEQ ID NO: 1 - DNA, 16 bp, Stevia rebaudiana
tttggtgacggtgcgg

SEQ ID NO: 2 - DNA, 17 bp, Stevia rebaudiana
tggggccaacccaagtc

SEQ ID NO: 3 - DNA, 16 bp, Stevia rebaudiana
ggcctgcagctcttct

SEQ ID NO: 4 - DNA, 17 bp, Stevia rebaudiana
gcgtccccaactcgatc

SEQ ID NO: 5 - DNA, 15 bp, Stevia rebaudiana
ggcaagggctgccgc

(19) United States
(12) Patent Application Publication
(10) Pub. No.: US 2016/0338396 A1
(43) Pub. Date: Nov. 24, 2016

(54) High Rebaudioside-A Plant and Methods of Producing the Same and Uses Thereof

(71) Applicant: GLG Life Tech Corporation, Vancouver (CA)

(72) Inventor: Qibin Wang, Bengbu City (CN)

(21) Appl. No.: 15/006,017

(22) Filed: Jan. 25, 2016

Related U.S. Application Data
(63) Continuation-in-part of application No. 13/977,548, filed on Sep. 6, 2013, now abandoned.

(30) Foreign Application Priority Data
Dec. 31, 2010 (CN) 201010618230.6
Dec. 31, 2010 (CN) 201010618515.X

(51) Int. Cl. A23L 27/10; A01H 1/02; A23L 2/60; A01H 5/12

(52) U.S. Cl. CPC A23L 27/11; A01H 5/12; A01H 1/02; A23L 2/60; A23V 2002/00

(57) ABSTRACT

A new stevia variety is characterized by remarkably high levels of Rebaudioside A (RA), and developed by the use of non-GMO selective breeding technologies and wherein such variety is uniquely identified by RAPD gene analysis, and comprises at least about 7-20% by weight RA in the leaf (leaf content), and comprises about 15-28% by weight total steviol glycosides in the leaf.

### CROSS-REFERENCE TO RELATED APPLICATIONS

The present application is a continuation-in-part of U.S. application Ser. No. 13/977,548, entitled “High Rebaudioside—A Plant and Methods of Producing the Same and Uses Thereof,” filed Sep. 6, 2013, which is a National Phase of International Patent Application Serial No. PCT/CA2012/000010, entitled “High Rebaudioside—A Plant and Methods of Producing the Same and Uses Thereof,” filed on Jan. 3, 2012, which in turn, claims priority to Chinese Patent Application Nos. 201010618230.6 and 201010618515.x, both filed on Dec. 31, 2010, the entire contents of each of which are hereby incorporated by reference in their entirety for all purposes.

### FIELD OF THE INVENTION

The present invention relates generally to methods of producing elite Stevia rebaudiana and particularly to a methods for improving the content of Rebaudioside A in Stevia rebaudiana.

### BACKGROUND

In the food and beverage industry, there is a general preference for the consumption of sweet foods, and manufacturers and consumers commonly add sugar in the form of sucrose (table sugar), fructose or glucose to beverages, food, etc. to increase the sweet quality of the beverage or food item. Although most consumers enjoy the taste of sugar, sucrose, fructose and glucose are high calorie sweeteners. Many alternatives to these high calorie sweeteners are artificial sweeteners or sugar substitutes, which can be added as an ingredient in various food items.

Common artificial sweeteners include saccharin, aspartame, and sucralose. Unfortunately, these artificial sweeteners have been associated with negative side effects. Therefore, alternative, natural non-caloric or low-caloric or reduced caloric sweeteners have been receiving increasing demand as alternatives to the artificial sweeteners and the high calorie sweeteners comprising sucrose, fructose and glucose. Like some of the artificial sweeteners, these alternatives provide a greater sweetening effect than comparable amounts of caloric sweeteners; thus, smaller amounts of these alternatives are required to achieve a sweetness comparable to that of sugar. These alternative, natural sweeteners, however, can be expensive to produce and/or possess taste characteristics different than sugar (such as sucrose), including, in some instances, undesirable taste characteristics such as sweetness linger, delayed sweetness onset, negative mouth feels and different taste profiles, such as off-tastes, including bitter, metallic, cooling, astringent, licorice-like tastes.

Steviol glycosides are responsible for the sweet taste of the leaves of the stevia plant (Stevia rebaudiana Bertoni). These compounds range in sweetness from 40 to 300 times sweeter than sucrose. They are heat-stable, pH-stable, and do not ferment.¹ They also do not induce a glycemic response when ingested, making them attractive as natural sweeteners to diabetics and others on carbohydrate-controlled diets. ¹Brandle, Jim (Aug. 8, 2004). “FAQ—Stevia, Nature's Natural Low Calorie Sweetener”. Agriculture and Agri-Food Canada. Retrieved Nov. 11, 2006.

The chemical structures of the diterpene glycosides of Stevia rebaudiana Bertoni are presented in FIG. 1. The physical and sensory properties are well studied generally only for Stevioside (STV) and Rebaudioside A. The sweetness potency of Stevioside is around 210 times higher than sucrose, Rebaudioside A in between 200 and 400 times, and Rebaudioside C and Dulcoside A around 30 times. Rebaudioside A is considered to have most favorable sensory attributes of the four major steviol glycosides (see Table 1):

| | | | | [a]²⁵_{D} | | | |
|---|---|---|---|---|---|---|---|
| | | ^{T}Meln | Mol. | (H₂O, | Solubility | Relative | Quality of |
| Name | Formula | ° C. | Weight | 1%, w/v) | in water, % | sweetness | taste |
| Steviol | C₂₀H₃₀O₃ | 212-213 | 318.45 | ND | ND | ND | Very bitter |
| Steviolmonoside | C₂₆H₄₀O₈ | ND | 480.58 | ND | ND | ND | ND |
| Stevioside | C₃₈H₆₀O₁₈ | 196-198 | 804.88 | −39.3 | 0.13 | 210 | Bitter |
| Rebaudioside A | C₄₄H₇₀O₂₃ | 242-244 | 967.01 | −20.8 | 0.80 | 200-400 | Less Bitter |
| Rebaudioside B | C₃₈H₆₀O₁₈ | 193-195 | 804.88 | −45.4 | 0.10 | 150 | Bitter |
| Rebaudioside C | C₄₄H₇₀O₂₂ | 215-217 | 951.01 | −29.9 | 0.21 | 30 | Bitter |
| Rebaudioside D | C₅₀H₈₀O₂₈ | 248-249 | 1129.15 | −29.5 | 1.00 | 220 | Like sucrose |

Stevia rebaudiana, after extraction and refinement is extensively used in the fields of foods, beverages, alcoholic liquor preparation, medicines, cosmetics, etc. In recent years, Stevia rebaudiana glycosides as extracts of Stevia rebaudiana have been used even more popularly as natural sweeteners and attractive alternatives to artificial sweeteners. They have become an excellent sweetening option since their caloric value is extremely low and they do not cause adverse effects to dental patients and diabetic patients. The potential market is huge.

Stevia rebaudiana glycosides mainly comprise the following nine components: Stevioside (STY, Rebaudioside A (RA), rubusoside, dulcoside A (DA), Rebaudioside C (RC), Rebaudioside F (RF), Rebaudioside D (RD), Steviolbioside (STB), and Rebaudioside B (RB).

The diterpene known as steviol is the aglycone of stevia's sweet glycosides, which are constructed by replacing steviol's carboxyl hydrogen atom with glucose to form an ester, and replacing the hydroxyl hydrogen with combinations of glucose and rhamnose to form an ether. The two primary compounds, stevioside and rebaudioside A, use only glucose: Stevioside has two linked glucose molecules at the hydroxyl site, whereas rebaudioside A has three, with the middle glucose of the triplet connected to the central steviol structure.

In terms of weight fraction, the four major steviol glycosides found in the stevia plant tissue are:
5-10% stevioside (STV) (250-300× of sugar)
2-12% rebaudioside A (RA)—most sweet (350-450× of sugar) and least bitter
1-2% rebaudioside C (RC)
½-1% dulcoside A. (DA)

Rebaudioside B, D, E and steviolbioside (STB) are known to be present in minute quantities;

Stevia diterpene glycosides, have a single base—steviol—and differ by the presence of carbohydrate residues at positions C₁₃ and C₁₉. These glycosides accumulate in Stevia leaves and compose approximately 10%-20% of the total dry weight. Typically, on a dry weight basis, the four major glycosides found in the leaves of Stevia are Dulcoside A (0.3%), Rebaudioside C (0.6%), Rebaudioside A (3.8%) and Stevioside (9.1%). Other glycosides identified in Stevia extract include Rebaudioside B, C, D, E, F, and M, Steviolbioside and Rubusoside. Among steviol glycosides only Stevioside and Rebaudioside A are currently widely available in commercial scale. The present Applicant has produced Reb C commercially over the last two to three years, although the low incidence in the leaf has limited the scale (and exacerbated the price) at which this extract could bring it to market, thus creating a disadvantage

The tastes of these components are different from one another and meet different demands of different consumer populations; for example, the consumers in the United States of America and Canada are fond of RA, whereas the consumers in Japan and Korea are fond of STV.

Currently, the marketed Stevia rebaudiana glycoside products are mainly RA and STV, and there are still no products mainly containing RD and/or RB, therefore, the methods for extracting Stevia rebaudiana glycoside also mainly focus on the purification and refinement of RA and STV.

A process for the general recovery of diterpene glycosides, including stevioside from the Stevia rebaudiana plant is described (U.S. Pat. No. 4,361,697). A variety of solvents, having different polarities, were used in a sequential treatment that concluded with a high performance liquid chromatographic (HPLC) separation procedure.

The method for the recovery of RA from the leaves of Stevia rebaudiana plants is provided in U.S. Pat. No. 4,082,858. Final purification is achieved by liquid chromatography subsequent followed by an initial extraction with water an alkanol having from 1 to 3 carbon carbons, preferably methanol. It is also disclosed that water may be used as the initial solvent, although the preferred solvent at this stage is a liquid haloalkane having from 1 to 4 carbon atoms. The preferred second solvent is an alkanol having from 1 to 3 carbon atoms, while the preferred third solvent is an alkanol having from 1 to 4 carbon atoms and optionally minor amounts of water.

U.S. Pat. No. 4,892,938, to Giovanetto discloses a purification process in which the aqueous extracts of the plant are purified by passing these aqueous extracts through a series of ion-exchange resins which are selected to remove various impurities. The sweet glycosides remain in the water and are recovered by evaporation of the water. The advantage is that everything is done in water, while most other processes involve the use of a solvent at some point. The disadvantage is that the final product is quite impure with only about 70% is a mixture of the sweet glycosides.

The balance is mainly material more polar than the sweet glycosides which we have identified as a complex mixture of polysaccharides (about 25%), and a small amount of yellow, oily material less polar than the sweet glycosides (about 5%).

The sweet glycosides obtained from Giovanetto process are always a mixture: namely the two principle sweet glycosides Stevioside (STV) and RA and the two minor sweet glycosides Dulcoside and RC.

It is generally accepted that Stevioside has an aftertaste which is undesirable. This aftertaste is present in Stevioside samples of even greater than 99% purity. On the other hand, RA does not possess an aftertaste and has a sweetness flavour comparable to sucrose. Thus, it is recognized as having the most desirable sensory properties of all the stevia glycosides. In addition to this complexity, various impurities are also present and some of these possess undesirable flavors. The entire matter is further clouded by the extreme difficulty of doing analyses.

RA has sweetness of a good quality and a degree of sweetness of 1.3 to 1.5 time that of stevioside and as such, it is most desirable to produce a plant with as high an RA content as possible. Furthermore, it is desirable to reduce the production cost of RA, to maintain the stable yield of dried leaves, to develop a variety of stevia which contains a high content amount of RA having excellent sweetening quality as a sweetening raw material, and at the same time, to maintain its continuous supply and to produce an excellent sweetener based on these.

There are two planting methods used for Stevia rebaudiana in China: the first one is the asexual propagation method, which is advantageous in that it can maintain the purity and superior quality of a variety. The disadvantages of this methods are that it is time-consuming, labour-intensive, and expensive when propagating wintering seminal seedlings in Autumn, keeping wintering seminal roots, and propagating cultivation seedlings in Spring. The overall production costs are high. Furthermore, irrespective of the relatively high content of RA in existing cultivars, the yield of leaves thereof is low and there is serious hybridity of varieties.

The second planting method is based upon sexual propagation. This is indeed time-saving, labour-saving and money-saving and has lower production costs as compared with the asexual propagation method. But the disadvantage of this propagation method is that the varieties are liable to degeneration. At the beginning of seed introduction for sexual propagation, the content of total Stevia rebaudiana glycoside was above 10%, but later the content of total Stevia rebaudiana glycoside falls to about 6%.

It can clearly be seen that both of the existing propagation methods are not desirable, as is. The patent document of the Chinese patent with publication number CN1327720A published on Dec. 26, 2001 discloses a breeding method for hybridized seeds of Stevia rebaudiana, of which the main content was using a sexual variety as male parent and an asexual cuttage variety as female parent to carry out hybridization, and the seeds of the female parent were collected. However, the hybridized seeds produced were not desirable since the quality of the sexual male parents was not stable and the asexual female parents did not undergo optimized selection. This method can meet neither the requirements of growers and processing enterprises nor the requirements of the industries of foods, beverages, medicines, cosmetics, and the like. Chinese patent CN1985575A of which the publication date is Jun. 27, 2007 discloses a method for systematic breeding of male parents and female parents of a cloned line of Stevia rebaudiana for cultivating new hybrid varieties, but in this method for seed breeding only one population hybridization was done, the F₁ generation hybridized seeds were harvested in a mixed way, therefore, there is still the undesirability of unstable traits. Therefore, to breed a novel elite variety of Stevia rebaudiana is not only a technical problem which urgently needs to be solved in planting and processing Stevia rebaudiana, but also an important technical issue in finding a healthy sugar source thereby meeting the demand for this sought after glycoside.

It is an object of the present invention to obviate or mitigate the above disadvantages.

### SUMMARY OF THE INVENTION

The present invention provides a new stevia variety characterized by remarkably high levels of Rebaudioside A (RA), developed by the use of non-GMO selective breeding technologies and wherein such variety is identified herein by RAPD gene analysis, thereby differentiating this new variety from Stevia plants of other varieties. Generally, conventional Stevia leaf comprises RA concentrations of about 6% by dry weight. The Stevia rebaudiana Bertonia variety of the present invention comprises from 7-20% by dry weight RA in the leaf (leaf content) (in one aspect 16%) and from about 15-22% total steviol glycosides content (TSG) in leaf (leaf content), (in one aspect 21%) both of which are exceptionally high. Generally, conventional Stevia leaf comprises TSG concentrations of about 10-11% by dry weight. Furthermore, the Stevia rebaudiana Bertonia variety of the present invention comprises, of the leaf total steviol glycosides content, a range of RA from 60-80% (in one aspect 76%) This can be compared to the amount of RA conventionally known of about 50%, a huge improvement

The present invention further provides a Stevia rebaudiana plant that comprises an Rebaudioside C leaf content of over 6% by weight, or at least 10% by weight or at least 15% by weight. The present invention further provides a Stevia rebaudiana plant that comprises an Rebaudioside C leaf content of from 7-20% by weight. The present invention further provides a Stevia rebaudiana plant that comprises an Rebaudioside C leaf content of from 16% by weight.

The present invention further provides a Stevia rebaudiana plant that comprises a TSG leaf content (dry weight) of 12-28%. The present invention further provides a Stevia rebaudiana plant that comprises a TSG leaf content of from 14-25% by weight. The present invention further provides a Stevia rebaudiana plant that comprises a TSG leaf content of 21% by weight.

The present invention further provides a Stevia rebaudiana plant that comprises an RA content (as a % of TSG, dry weight) of from 60-85%. The present invention further provides a Stevia rebaudiana plant that comprises an RA content (as a % of TSG, dry weight) of from 70-80%. The present invention further provides a Stevia rebaudiana plant that comprises an RA content (as a % of TSG, dry weight) of about 76%.

The present invention provides varieties of Stevia rebaudiana which are high in RA, a means to genetically distinguish such varieties, and methods to maintain the characteristics thereof, thereby differentiating them from Stevia plants of other varieties, and sweetener compositions comprising extracts of the plant varieties of the present invention.

The present invention further provides a Stevia rebaudiana plant that comprises an Rebaudioside A leaf content of from greater than 6% by weight, and a total steviol glycosides leaf content of at least 15-28% by weight and comprises three bands, one between each of i) 500 bp-750 bp, ii) 750-1000 bp; and iii) about 2000 bp, when analyzed by Random Amplified Polymorphic DNA (RAPD) using the primers of SEQ ID NO: 1, SEQ ID NO: 2, SEQ ID NO: 3 and SEQ ID NO: 4.

The present invention further provides a Stevia rebaudiana plant that comprises an Rebaudioside A leaf content of from greater than 7-20% by weight, and a total steviol glycosides leaf content of at least 15-28% by weight and comprises three bands, one between each of i) 500 bp-750 bp, ii) 750-1000 bp; and iii) about 2000 bp, when analyzed by Random Amplified Polymorphic DNA (RAPD) using the primers of SEQ ID NO: 1, SEQ ID NO: 2, SEQ ID NO: 3 and SEQ ID NO: 4.

The present invention further provides a Stevia rebaudiana plant that comprises an Rebaudioside A leaf content of from greater than 10% by weight, and a total steviol glycosides leaf content of at least 15-28% by weight and comprises three bands, one between each of i) 500 bp-750 bp, ii) 750-1000 bp; and iii) about 2000 bp, when analyzed by Random Amplified Polymorphic DNA (RAPD) using the primers of SEQ ID NO: 1, SEQ ID NO: 2, SEQ ID NO: 3 and SEQ ID NO: 4.

The present invention further provides a Stevia rebaudiana plant that comprises an Rebaudioside A leaf content selected from:

i) greater than 6% by weight;

ii) greater than 10% by weight;

iii) greater than 15% by weight;

iv) around 16% by weight;

and a total steviol glycosides leaf content selected from the group consisting of:

i) 15-28% by weight;

ii) 14-25% by weight;

iii) 18-23% by weight;

iv) around 21% by weight;

and comprises three bands, one between each of i) 500 bp-750 bp, ii) 750-1000 bp; and iii) about 2000 bp, when analyzed by Random Amplified Polymorphic DNA (RAPD) using the primers of SEQ ID NO: 1, SEQ ID NO: 2, SEQ ID NO: 3 and SEQ ID NO: 4.

The present invention further provides a Stevia rebaudiana plant that comprises an Rebaudioside A leaf content selected from the group consisting of:

i) greater than 6% by weight;

ii) greater than 10% by weight;

iii) greater than 15% by weight;

iv) around 16% by weight;

and a total steviol glycosides leaf content selected from the group consisting of:

i) 15-28% by weight;

ii) 14-25% by weight;

iii) 18-23% by weight;

iv) around 21% by weight;

and a Rebaudioside A content as a percentage of total steviol glycosides selected from the group consisting of:

i) 60-85% by weight;

ii) 70-80% by weight;

iii) around 76% by weight;

and comprises three bands, one between each of i) 500 bp-750 bp, ii) 750-1000 bp; and iii) about 2000 bp, when analyzed by Random Amplified Polymorphic DNA (RAPD) using the primers of SEQ ID NO: 1, SEQ ID NO: 2, SEQ ID NO: 3 and SEQ ID NO: 4.

The object of the present invention is to overcome the disadvantages of existing varieties of Stevia rebaudiana, to breed a novel elite variety of Stevia rebaudiana with high yield of leaves, high content of total Stevia rebaudiana glycoside, high content of RA, strong resistance (i.e, “three high and one resistance”), and stable traits. It has been surprisingly found that this variety not only comprises higher than conventional RA but also concomitantly higher than expected RC. RC and RA are the two best tasting glycosides in the Stevia leaf.

To realize the object described above, the bases for selection breeding of elite variety of Stevia rebaudiana in accordance with the present invention is based on the following 1) Stevia rebaudiana has the trait of being capable of both sexual propagation and asexual propagation; 2) asexual propagation may be used to stabilize superior traits; and 3) heterosis.

In one aspect, the present invention discloses a method for breeding Stevia rebaudiana with a high content of RA, which comprises the following steps: selecting a plant with a RA content in the leaf of at least 6% by weight (“core plant”), asexually reproducing the core plant to produce parent plants, hybridizing the parent plants to produce F₁ generation seeds, and stabilizing the traits of the F₁ generation (namely, in the leaves of the F₁ generation, producing an RA content of greater than 6% by weight and thereafter producing F₂ generation seeds by a backcross method. The present invention has the advantages of high yield of leaves, high content of total glycoside, high content of rebaudioside A (RA), strong resistance, and stable traits of plants.

In a further aspect, the present invention provides a method of producing via breeding a Stevia rebaudiana elite variety with a high content of RA which comprises the steps of:
(1) selecting a plant with a RC content in the leaf of greater than 6% by dry weight (“core plant”);
(2) reproducing the core plant to produce parent plants;
(3) hybridizing parent plants to produce F₁ generation seeds;
(4) stabilizing the traits of the F₁ generation (namely, in the leaves of the F₁ generation, an RA content of at least 7-20% by dry weight;
(3) producing F₂ generation seeds by a backcross method; and
(4) producing F₃ generation seeds by a backcross method.

Stevia rebaudiana elite variety seeds, cells, plants, germplasm, breeding lines, varieties, and plant parts produced by these methods and/or derived from variety provided herein are within the scope of the invention.

The present invention further provides a natural sweetener composition comprising compositions comprising RA extracted and purified from any of the plant material as described herein.

The present invention further provides a natural flavour composition comprising RA extracted and purified from any of the plant material as described herein.

The present invention further provides foods, beverages, nutraceuticals, functional foods, medicinal formulations, cosmetics, health products, condiments and seasonings comprising RA extracted and purified from any of the plant material as described herein.

The present invention further provides a natural sweetener composition comprising a blend of RA extract along with one or both of Stevioside (STV) extract and Rebaudioside C extract wherein the relative weight percent of Rebaudioside A is higher than in known extracted compositions.

The natural sweetener compositions of the present invention may be zero calories or merely reduced calorie, as desired.

The present invention further provides a purification process for extracting, from the Stevia rebaudiana elite variety, described herein, a composition comprising a blend of Rebaudioside A extract along with one or both of Stevioside (STV) extract and Rebaudioside C extract wherein the relative weight percent of Rebaudioside A is higher than in known extracted compositions.

What the present invention provides are compositions of specific and selected steviol glycosides which achieve benefits and advantages above and beyond the use of each extracted glycoside alone. These natural sweetener compositions have a taste profile comparable to sugar, are desired, are not prohibitively expensive to produce and can be added, for example, to beverages and food products to satisfy consumers looking for a sweet taste. As such, these compositions allow for the customization of sweetening goals.

What has been created within the scope of the invention is a new Stevia rebaudiana elite variety which will significantly reduce the cost of producing high purity RA extracts by approximately 50-60%. This new varietal comprises about double the amount of TSG and nearly triple of the amount of RA as compared to stevia leaf currently available on the market today. With this surprisingly enhanced RA content, producing a ton of either intermediate or high purity extract will require significantly less stevia leaf (the predominant cost factor in manufacturing) as compared to presently used leaves. Other costs of productions will be likewise reduced. The leave of the present varietal, so rich in RA, is the first of its kind

These and other objects and advantages of the present invention will become more apparent to those skilled in the art upon reviewing the description of the preferred embodiments of the invention, in conjunction with the figures and examples. A person skilled in the art will realize that other embodiments of the invention are possible and that the details of the invention can be modified in a number of respects, all without departing from the inventive concept. Thus, the following drawings, descriptions and examples are to be regarded as illustrative in nature and not limiting.

### BRIEF DESCRIPTION OF THE DRAWINGS

Embodiments of the present invention will now be described, by way of example only, with reference to the attached Figures, wherein:

FIG. 1 is ISSR fingerprint of both parents and seven accessions;

FIG. 2 is ISSR fingerprint of both parents and seven accessions;

FIG. 3 is ISSR fingerprint of both parents and seven accessions

FIG. 4 is ISSR fingerprint of both parents and seven accessions;

FIG. 5 is ISSR fingerprint of both parents and seven accessions;

FIG. 6 is a Phenogram, generated using UPGMA, of all seven accessions of Stevia rebaudiana based on ISSR data;

FIG. 7 are ISSR fingerprints of Stevia H2, H3, H4 and H5 DNA molecular identification report from AT Lab;

FIG. 8 is a flow diagram of the extraction process for extracting a primary extract of steviol glycosides from the leaves of Stevia rebaudiana to yield a mother liquor from which RA may be extracted and purified;

FIG. 9 is a graphic representation of the chemical structure of RA;

FIG. 10 is an electrophoresis diagram of the DNA base sequences of the Morita variety M wherein the characteristic base sequences are indicated with arrows 1=GLG-High-RC cultivar and M=DM 2000 plus Marker—(SEQ ID NO 5) Primer 1: 5′-GGCAAGGGCTGCCGC-3′;

FIG. 11 is an electrophoresis diagram of the DNA base sequences wherein the characteristic base sequences are indicated with arrows and wherein 1=GLG-High-RC cultivar and M=DM 2000 plus Marker and wherein (SEQ ID NO 1) Primer 2: 5′-TTTGGTGACGGTGCGG-3′;

FIG. 12 is an electrophoresis diagram of the DNA base sequences wherein the characteristic base sequences are indicated with arrows and wherein 1=GLG-High-RC cultivar and M=DM 2000 plus Marker and wherein (SEQ ID NO 2) Primer 3: 5′-TGGGGCCAACCCAAGTC-3′;

FIG. 13 is an electrophoresis diagram of the DNA base sequences wherein the characteristic base sequences are indicated with arrows and wherein 1=GLG-High-RC cultivar and M=DM 2000 plus Marker and wherein (SEQ ID NO 3) Primer 4: 5′-GGCCTGCAGCTCTTCT-3; and

FIG. 14 is an electrophoresis diagram of the DNA base sequences wherein the characteristic base sequences are indicated with arrows and wherein 1=GLG-High-RC cultivar and M=DM 2000 plus Marker and wherein (SEQ ID NO 4) Primer 5: 5′-GCGTCCCCAACTCGATC-3.

### DETAILED DESCRIPTION OF THE INVENTION

A detailed description of one or more embodiments of the invention is provided below along with accompanying figures that illustrate the principles of the invention. As such this detailed description illustrates the invention by way of example and not by way of limitation. The description will clearly enable one skilled in the art to make and use the invention, and describes several embodiments, adaptations, variations and alternatives and uses of the invention, including what we presently believe is the best mode for carrying out the invention. It is to be clearly understood that routine variations and adaptations can be made to the invention as described, and such variations and adaptations squarely fall within the spirit and scope of the invention.

In other words, the invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

Certain definitions used in the specification are provided below. Also in the examples which follow, a number of terms are used. In order to provide a clear and consistent understanding of the specification and claims, the following definitions are provided:

In the present disclosure and claims (if any), the word “comprising” and its derivatives including “comprises” and “comprise” include each of the stated integers or elements but does not exclude the inclusion of one or more further integers or elements. The term process may be used interchangeably with method, as referring to the steps of breeding (sexual and asexual) as described and claimed herein.

The term Rebaudioside A may be used interchangeably with RA (or Reb A), the term Rebaudioside C may be used interchangeably with RC (or Reb C) and the term Stevioside may be used interchangeably with STV. This type of latitude in usage applies to the description of all of glycosides.

For clarity, it is to be noted that “steviol glycosides” have been referred to as stevia, stevioside, and stevia glycoside in the scientific literature. Generally, the term, steviol glycosides has been adopted for the family of steviol derivatives with sweetness properties that are derived from the stevia plant. More recently, the term, stevia, is used more narrowly to describe the plant or crude extracts of the plant, while stevioside is the common name for one of the specific glycosides that is extracted from stevia leaves. Stevioside is distinct from steviolbioside.

As used herein, the term “about” in connection with a measured quantity, refers to the normal variations in that measured quantity, as expected by a skilled artisan making the measurement and exercising a level of care commensurate with the objective of measurement.

Breeding of New High Reb C Varietal

The present invention provides a new elite variety of Stevia rebaudiana with high yield of leaves, high content of total steviol glycosides, high content of RC, strong resistance (i.e., “three high and one resistance”), and stable traits. As used herein, the term “three high and one resistance” (high leaf yield, high glycoside content, and high specific steviol glycoside content (i.e., specifically, here, RC)); resistance refers to pesticide and plant disease.]

The process in which a breeder crosses a donor parent variety possessing a desired trait or traits to a recurrent parent variety (which is agronomically superior but lacks the desired level or presence of one or more traits) and then crosses the resultant progeny back to the recurrent parent one or more times is called “backcrossing”. Backcrossing can be used to introduce one or more desired traits from one genetic background into another background that is lacking the desired traits. As such, the manipulation of living organisms in the manner described in this application is called selective “breeding”.

The genetic manipulation of living organisms is called “breeding”.

As used herein, the term “plant” includes reference to an immature or mature whole plant, including a plant from which seed or grain or anthers have been removed. Seed or embryo that will produce the plant is also considered to be the plant. As used herein, the term “plant parts” includes leaves, stems, roots, root tips, anthers, seed, grain, embryo, pollen, ovules, flowers, cotyledon, hypocotyl, pod, flower, shoot, stalk, tissue, cells and the like.

“Plant reproduction” is the production of new individuals or offspring in plants, which can be accomplished by “sexual” or “asexual” means. Sexual reproduction produces offspring by the fusion of gametes, resulting in offspring genetically different from the parent or parents. Asexual reproduction produces new individuals without the fusion of gametes, genetically identical to the parent plants and each other, except when mutations occur. In seed plants, the offspring can be packaged in a protective seed, which is used as an agent of dispersal.

Sexual reproduction involves creation of a new individual produced by the combining features or genes from two parents. Sexual reproduction in plants generally occurs through the medium of flowers. The flower structure is made up of pollen producing male part known as stamen, and female part called pistil that contains the ovary and eggs. Pollination is the process that starts the sexual reproducing mechanism. The petals play a vital role in attracting insects to the flowers that carry pollen from one plant to another. Wind pollination occurs where flowers do not have petals. Pollination results in production of seeds, and almost all plants are reproduced through this mechanism.

Sexual reproduction involves two fundamental processes: meiosis, which rearranges the genes and reduces the number of chromosomes, and fertilization, which restores the chromosome to a complete diploid number. In between these two processes, different types of plants and algae vary, but many of them, including all land plants, undergo alternation of generations, with two different multicellular structures (phases), a gametophyte and a sporophyte.

The gametophyte is the multicellular structure (plant) that is haploid, containing a single set of chromosomes in each cell. The gametophyte produces male or female gametes (or both), by a process of cell division called mitosis. In vascular plants with separate gametophytes, female gametophytes are known as megagametophytes (mega=large, they produce the large egg cells) and the male gametophytes are called microgametophytes (micro=small, they produce the small sperm cells). The fusion of male and female gametes (fertilization) produces a diploid zygote, which develops by mitotic cell divisions into a multicellular sporophyte. The mature sporophyte produces spores by meiosis, sometimes referred to as “reduction division” because the chromosome pairs are separated once again to form single sets.

As used herein the term asexual reproduction means any reproductive process that does not involve meiosis or syngamy is said to be asexual, or vegetative. The absence of syngamy means that such an event can occur in the sporophyte generation or the gametophyte stage. Because of the lack of new genetic material, an organism clones itself through this process and makes genetically identical organisms.

The most common form of plant reproduction utilized by people is seeds, but a number of asexual methods are utilized which are usually enhancements of natural processes, including: cutting, grafting, budding, layering, division, sectioning of rhizomes or roots, stolons, tillers (suckers) and artificial propagation by laboratory tissue cloning. Asexual methods are most often used to propagate cultivars with individual desirable characteristics that do not come true from seed. Fruit tree propagation is frequently performed by budding or grafting desirable cultivars (clones), onto rootstocks that are also clones, propagated by layering.

In horticulture, a “cutting” is a branch that has been cut off from a mother plant below an internode and then rooted, often with the help of a rooting liquid or powder containing hormones. When a full root has formed and leaves begin to sprout anew, the clone is a self-sufficient plant, genetically identical to the mother plant. Examples include cuttings from the stems

of blackberries (Rubus occidentalis), Africanviolets (Saintpaulia), verbenas (Verbena) to produce new plants. A related use of cuttings is grafting, where a stem or bud is joined onto a different stem. Nurseries offer for sale trees with grafted stems that can produce four or more varieties of related fruits, including apples. The most common usage of grafting is the propagation of cultivars onto already rooted plants, sometimes the rootstock is used to dwarf the plants or protect them from root damaging pathogens. Since vegetatively propagated plants are clones, they are important tools in plant research.

For crosses you have parents (P) and offspring (Filial generations) F1=children of parents, F2=grandchildren, F3=great grandchildren, etc. . . .

As used herein, “heterosis”, or hybrid vigor, or outbreeding enhancement, is the improved or increased function of any biological quality in a hybrid offspring. The adjective derived from heterosis is heterotic. Heterosis is the occurrence of a superior offspring from mixing the genetic contributions of its parents. These effects can be due to Mendelian or inheritance. The physiological vigor of an organism as manifested in its rapidity of growth, its height and general robustness, is positively correlated with the degree of dissimilarity in the gametes by whose union the organism was formed. The more numerous the differences between the uniting gametes—at least within certain limits—the greater on the whole is the amount of stimulation.

Heterosis is the opposite of inbreeding depression. Inbreeding depression leads to offspring with deleterious traits due to homozygosity. The inverse of heterosis, when a hybrid inherits traits from its parents that are not fully compatible, with deleterious results, is outbreeding depression. Crosses between inbreds from different heterotic groups result in vigorous F1 hybrids with significantly more heterosis than F1 hybrids from inbreds within the same heterotic group or pattern. Heterotic groups are created by plant breeders to classify inbred lines, and can be progressively improved by reciprocal recurrent selection.

Polymerase chain reaction (PCR)-based RAPD (random amplified polymorphic DNA method) or ISSR (inter-simple sequence repeat) variations as phylogenetic markers for investigating relationships among plants has been clearly established (Morgante and Olivieri 1993²; Ghislain et al. 1999³). As such, both RAPD- and ISSR-fingerprinting data may be used herein, alone and in combination, to examine the level of genetic diversity within the uniquely bred Stevia cultivars. ²Morgante, M., and Olivieri, A. M. 1993. PCR-amplified microsatellites as markers in plant genetics. Plant J. 3: 175-182.³Gupta, M., Chyi, Y. S., Romero-Severson, J., and Own, J. L. 1994.Amplification of DNA markers from evolutionarily diverse genomes using single primers of simple-sequence repeats. Theor. Appl. Genet. 89: 998-1002

The separation of macromolecules in an electric field is called electrophoresis. A very common method for separating proteins by electrophoresis uses a discontinuous polyacrylamide gel as a support medium and sodium dodecyl sulfate (SDS) to denature the proteins. The method is called sodium dodecyl sulfate polyacrylamide gel electrophoresis (SDS-PAGE). The most commonly used system is also called the Laemmli method. SDS (also called lauryl sulfate) is an anionic detergent, meaning that when dissolved its molecules have a net negative charge within a wide pH range. A polypeptide chain binds amounts of SDS in proportion to its relative molecular mass. The negative charges on SDS destroy most of the complex structure of proteins, and are strongly attracted toward an anode (positively-charged electrode) in an electric field.

Polyacrylamide gels restrain larger molecules from migrating as fast as smaller molecules. Because the charge-to-mass ratio is nearly the same among SDS-denatured polypeptides, the final separation of proteins is dependent almost entirely on the differences in relative molecular mass of polypeptides. In a gel of uniform density the relative migration distance of a protein (Rf, the f as a subscript) is negatively proportional to the log of its mass. If proteins of known mass are run simultaneously with the unknowns, the relationship between Rf and mass can be plotted, and the masses of unknown proteins estimated.

Protein separation by SDS-PAGE can be used herein to estimate relative molecular mass, to determine the relative abundance of major proteins in a sample, and to determine the distribution of proteins among fractions. The purity of protein samples can be assessed and the progress of a fractionation or purification procedure can be followed. Specialized techniques such as Western blotting, two-dimensional electrophoresis, and peptide mapping can be used to detect gene products, to find similarities among them, and to detect and separate isoenzymes of proteins.

The RAPD method used for the identification in the present invention is one of the analytical methods of DNA, and it is a method for the analysis by electrophoresis of a DNA pattern amplified in a DNA region sandwiched between the same or similar sequences as or to the primers used in a PCR reaction (Polymerase chain reaction) using a plural number of primers. In addition, for cetyl trimethyl ammonium bromide (CTAB) is a quaternary ammonium salt having a long chain alkyl group, and it forms an insoluble complex with a poly anion such as nucleic acid, it can be utilized for isolating a nucleic acid.

In the means by which to classify a variety based on differences in DNA, a genome DNA is singly isolated from a plant by CTAB, ribonucleic acid (RNA) is removed, and a PCR amplified product obtained by the PCR method by use of a primer mix is distinguished by the differences in DNA finger print obtained by the agarose gel electrophoresis method.

Varieties which contain a relatively high concentration of Rebaudioside A are crossbred, and selected, such being the goal of the breeding methods described and claimed herein.

As provided herein, “higher” or “high” RA refers to a greater RA content, in the novel varietals described and claimed herein as compared to the wild type Stevia rebaudiana. At least, this refers to plant, more specifically leaves, from which are extracted a composition or blend, with greater than 6% RA by dry leaf weight. In another aspect, this refers to greater than 10% RA by dry leaf weight. In another aspect, this refers to greater than 15% RA by dry leaf weight. In another aspect, this refers to 7-20% RA by dry leaf weight. In another aspect, this refers to about 16% RA by dry leaf weight.

As provided herein, “higher” or “high” total steviol glycosides (or glycoside) content (TSG) refers to a greater total TSG content in the leaf, in the novel varietals described and claimed herein as compared to the wild type Stevia rebaudiana. At least, in the context of the invention, this refers to at least 15-28% TSG in the leaf. In another aspect, this refers to 14-25% TSG in the leaf. In another aspect, this refers to about 21% TSG in the leaf.

As provided herein, within the step of “selecting the plants in the perfect stage with a high RA content as parents and hybridizing them to produce F₁ generation seeds”, perfect refers to the desired level of RA in parental plant stock at a given stage. For example, in selecting the core parent, this refers to greater than 6% by weight RA in the leaf. Within successive propagation and then hybridization programs as provided herein, the base level of RA has been found to successively increase to the level of 7-20% RC and, in one aspect (although not exclusively) at least 10% RA in the leaf, and in another aspect, at least 15% RA in the leaf. More preferably, it refers to a target of about 16% RA in the leaf.

In the following, the breeding process, the characteristics thereof, etc will be specifically described. However, the present invention is not limited to these breeding processes and cultivating methods.

Within the scope of the invention, the varieties have shown uniformity and stability for all traits, as described in the following variety description information. They have been self-pollinated a sufficient number of generations, with careful attention to uniformity of plant type to ensure a sufficient level of homozygosity and phenotypic stability. The varieties have been increased with continued observation for uniformity. No variant traits have been observed or are expected.

Genetic Marker Profile

In addition to phenotypic observations, a plant can also be identified by its genotype. The genotype of a plant can be characterized through a genetic marker profile which can identify plants of the same variety or a related variety, or which can be used to determine or validate a pedigree. Genetic marker profiles can be obtained by techniques such as restriction fragment length polymorphisms (RFLPs), RAPDs, arbitrarily primed polymerase chain reaction (AP-PCR), DNA amplification fingerprinting (DAF), sequence characterized amplified regions (SCARs), amplified fragment length polymorphisms (AFLPs), simple sequence repeats (SSRs) also referred to as microsatellites, or single nucleotide polymorphisms (SNPs).

Particular markers used for these purposes are not limited to any particular set of markers, but are envisioned to include any type of marker and marker profile which provides a means of distinguishing varieties. The genetic marker profile is also useful in breeding and developing backcross conversions.

A backcross conversion occurs when DNA sequences are introduced through backcrossing. A backcross conversion may produce a plant with a trait or locus conversion in at least two or more backcrosses, including at least 2 backcrosses, at least 3 backcrosses, at least 4 backcrosses, at least 5 backcrosses, or more. Molecular marker assisted breeding or selection may be utilized to reduce the number of backcrosses necessary to achieve the backcross conversion. For example, see Openshaw, S. J. et al., Marker-assisted Selection in Backcross Breeding. In: Proceedings Symposium of the Analysis of Molecular Data, August 1994, Crop Science Society of America, Corvallis, Oreg., where it is demonstrated that a backcross conversion can be made in as few as two backcrosses.

The complexity of the backcross conversion method depends on the type of trait being transferred (a single gene or closely linked genes compared to unlinked genes), the level of expression of the trait, the type of inheritance (cytoplasmic or nuclear), dominant or recessive trait expression, and the types of parents included in the cross. It is understood by those of ordinary skill in the art that for single gene traits that are relatively easy to classify, the backcross method is effective and relatively easy to manage. Desired traits that may be transferred through backcross conversion include, but are not limited to, sterility (nuclear and cytoplasmic), fertility restoration, nutritional enhancements, drought tolerance, nitrogen utilization, altered fatty acid profile, low phytate, industrial enhancements, disease resistance (bacterial, fungal or viral), insect resistance, and herbicide resistance.

The backcross conversion may result from either the transfer of a dominant allele or a recessive allele. Selection of progeny containing the trait of interest is accomplished by direct selection for a trait associated with a dominant allele. Selection of progeny for a trait that is transferred via a recessive allele requires growing and selfing the first backcross generation to determine which plants carry the recessive alleles. Recessive traits may require additional progeny testing in successive backcross generations to determine the presence of the locus of interest. The last backcross generation is usually selfed to give pure breeding progeny for the trait(s) being transferred, although a backcross conversion with a stably introgressed trait may also be maintained by further backcrossing to the recurrent parent with subsequent selection for the trait.

Along with selection for the trait of interest, progeny are selected for the phenotype of the recurrent parent. The backcross is a form of inbreeding, and the features of the recurrent parent are automatically recovered after successive backcrosses.

Pedigree breeding starts with the crossing of two genotypes having one or more desirable characteristics that is desired the two original parents do not provide all the desired characteristics, other sources can be included in the breeding population. In the pedigree method, superior plants are selfed and selected in successive filial generations. In the succeeding filial generations, the heterozygous allele condition gives way to the homozygous allele condition as a result of inbreeding. Successive filial generations of selfing and selection is practiced: F1, F2 and optionally F3. After such inbreeding, successive filial generations will serve to increase seed of the developed variety.

In addition to being used to create backcross conversion populations, backcrossing can also be used in combination with pedigree breeding. As discussed previously, backcrossing can be used to transfer one or more specifically desirable traits from one variety (the donor parent) to a developed variety (the recurrent parent), which has overall good agronomic characteristics yet lacks that desirable trait or traits. However, the same procedure can be used to move the progeny toward the genotype of the recurrent parent but at the same time retain many components of the non-recurrent parent by stopping the backcrossing at an early stage and proceeding with selfing and selection. For example, a soybean variety may be crossed with another variety to produce a first generation progeny plant. The first generation progeny plant may then be backcrossed to one of its parent varieties. Progeny are selfed and selected so that the newly developed variety has many of the attributes of the recurrent parent and yet several of the desired attributes of the donor parent.

Producing F₂ Generation Seeds b a Backcross Method

(1) Selection of Parents

Stevia rebaudiana is a cross-pollinated plant with self-sterility, so the genetic constitution of a Stevia rebaudiana population is always of a heterozygous type, the heredity of the sexual offspring is not easily stabilized, and both advantageous variations and harmful variations are kept at the same time, which presents a challenge to the selection of elite individual plants. According to the characteristics, features, growing periods, blooming periods, resistance and adaptability of individual plants in different growth periods of Stevia rebaudiani in each of the different planting areas in China and foreign countries, the authors observed and monitored them so as to select elite individual plants with high yield of leaves, high content of glycoside in the leaves and strong resistance, for isolated management.

(2) Hybridization in Perfect Stage to Produce Seeds
1. When selecting, matching, and combining from the elite individual plants selected in the current year and previous years, two elite individual plants with luxuriant growth, high yield of leaves, strong resistance, similar blooming periods, high seed-setting rate, and high content of total glycoside and RA were selected from as distant pedigrees as far as possible to carry out a combined hybridization test.
2. In the next year, the seeds which were harvested from plants in the current year according to combination and plants are sown in cultivar gardens, field observations were performed and recorded, the yield and content of leaves determined, and good combinations were selected from them as male parents and female parents.
3. According to the principle that asexual propagation can maintain superior traits, the male parents and female parents which were selected good combinations were subjected to asexual propagation so as to maintain their superior traits and form cloned lines of individual plants of male parents and female parents. The cloned lines of male parents and female parents of selected and matched good combinations were colonized at a preferred ratio of about 1:1 to carry out population hybridization of the two lines, and the F₁ generation hybridized seeds were harvested in a mixed way.

(3) Stabilization of Traits by Asexual Propagation

Individual plants with luxuriant growth, high yield of leaves, strong resistance, similar blooming periods, high seed-setting rate, and high content of total glycoside and RA were selected from the harvested F₁ generations by using the methods in the steps described above, and then asexual propagation was carried out to maintain their superior traits.

(4) Superior F₁ generations after their traits were stabilized were used as female parents and the original asexual male parents were used as male parents to carry out backcrossing, the male parents and female parents were colonized at a ratio preferably of about 1:3, and the F₂ generation seeds were harvested so as to obtain the target variety.

In addition to other characteristic described herein, it is preferred, as compared with other existing varieties, that the variety of the of the present invention has the following advantages:
1. The characteristics of hybridized F₂ Stevia rebaudiana are: upright branches and stems, lodging resistance, luxuriant growth and large and broad leaves.
2. The superior qualities of the novel hybridized F₂ Stevia rebaudiana variety are:
   high yield of dry leaves per Chinese acre which is greatly increased relative to that of other varieties;
   the total content of glycoside and content of RA are also greatly increased relative to those of sexual cultivars;
   high resistance, with a stronger disease resistance and a stronger pest resistance than other varieties.

One method for systematic breeding of male parents and female parents of cloned lines of Stevia rebaudiana for cultivating a novel elite variety as provided herein is based on the following linkages and discoveries:
Stevia rebaudiana has the trait of being capable of both sexual propagation and asexual propagation,
using asexual propagation to stabilize superior traits,
heterosis—the old Stevia rebaudiana varieties can be replaced with new ones successively and the quality thereof can be improved successively if the breeding goal of “three high and one resistance” is focused on and adhered to successively
novel combinations can be selected and novel Stevia rebaudiana varieties can be cultivated according to the demands of the market.

Since the cultivation of the novel hybridized varieties of Stevia rebaudiana is low in cost and superior in efficiency, all of the existing varieties of sexual cultivation and asexual cultivation would necessarily be partially or fully replaced by novel hybridized varieties of Stevia rebaudiana and generous economic benefits and social benefits would be created for the growers, the processing enterprises and the nation.

The particular embodiments of this aspect of the present invention will be illustrated hereinafter through the description of the process of the present invention.

Selection of Parents

Plants selected as “core plant” are those with one or more unusual growing traits (high, leaf size or dimension, number or leaves or other characteristics). The one or more core plants are tested immediately for RA leaf content, using techniques such as, for example, NIR and HPLC. The selected core plant is propagated and hybridized. The desire is to achieve elite individual plants with high yield of leaves, high RA content of steviol glycoside in leaves and strong resistance, for comparing with the traits of existing sexual and asexual plants; four plants (their serial numbers were 01, 02, 03 and 04, respectively) were selected to carry out isolated management; and each elite individual plant was tested 3 times and the mean values of the data were taken in order to ensure the reliability of the selected elite individual plants. (The results for comparing the contents and leaf yields of the 4 individual plants with those of existing sexual and asexual plants).

Hybridization in Perfect Stage to Produce Seeds
1. In the next year, the seeds which were harvested from the plant divisions of elite individual plants 01, 02, 03, and 04 were sown in cultivar gardens, field observations were performed and recorded, and the yields and contents of leaves were determined. (the results for comparing the contents and leaf yields of the 4 individual plants)
2. According to the theory that superior traits can be maintained by using asexual propagation, the good combinations of the elite individual plants 01, 02, 03, and 04 were subjected to asexual propagation so as to maintain their superior traits and form cloned lines of individual plants of male parents and female parents. The cloned lines of male parents and female parents of selected and matched good combinations were colonized at a ratio of preferably about 1:1, (plant number and plant distance) population hybridization of the two lines was carried out, and F₁ generation hybridized seeds were harvested in a mixed way. A certain quantity (number) of superior F₁ generations were selected, their seeds were sown in cultivar gardens, the mean values of RA content, total glycoside content and leaf yield were determined, then they were compared with the RA content, total glycoside content and leaf yield of male parents, female parents and hybridized F₁ generations, (comparison results) and it was found that all of the above indices of hybridized F₁ generations were significantly improved as compared with both male parents and female parents, and even more greatly improved as compared with existing sexual and asexual varieties.
3. The superior F₁ generations were subjected to asexual propagation and isolated management so as to stabilize their superior traits.
4. The superior F₁ generation hybridized seeds after being stabilized were used as female parents and the original asexual male parents were used as male parents to carry out backcrossing, the male parents and female parents were colonized at a ratio of preferably about 1:3 when producing seeds by backcrossing, and the F₂ generation seeds were harvested so as to obtain the target variety. A certain quantity (number) of superior F₂ generations were selected, the mean values of RA content, total glycoside content and leaf yield were determined, then they were compared with the RA content, total glycoside content and leaf yield of the original asexual male parents and the hybridized F₁ generations, (comparison results) and it was found that all of the above indices of hybridized F₂ generations were significantly improved as compared with the original asexual male parents and the F₁ generations, and even more greatly improved as compared with existing sexual and asexual varieties

Producing F₃ Generation Seeds by a Backcross Method

(1) Selection of Parents

Stevia rebaudiana is a cross-pollinated plant with self-sterility, so the genetic constitution of a Stevia rebaudiana population always of a heterozygous type, the heredity of the sexual offsprings is not easily stabilized, and both advantageous variations and harmful variations are kept at the same time, which bring a challenge and a possibility for us to select individual plants. According to the characteristics, features, growing periods, blooming periods, resistance and adaptability of individual plants in different growth periods of Stevia rebaudiani in each of the different planting areas in China and foreign countries, the authors observed and monitored them so as to select elite individual plants with high yield of leaves, high content of glycoside in the leaves and strong resistance, for isolated management.

(2) Hybridization in Perfect Stage to Produce Seeds
\textcircled{1} When selecting, matching, and combining from the elite individual plants selected in the current year and previous years, two elite individual plants with luxuriant growth, high yield of leaves, strong resistance, similar blooming periods, high seed-setting rate, and high content of total glycoside and RA were selected from distant pedigrees as far as possible to carry out a combined hybridization test.
\textcircled{2} In the next year, the seeds which were harvested from plants in the same year according to combination and plants were sown in cultivar gardens, field observations were performed and recorded, the yield and content of leaves determined, and variety combinations selected from them as male parents and female parents.
\textcircled{3} According to the discovery that asexual propagation can maintain superior traits, the male parents and female parents which were selected good combinations were subjected to asexual propagation so as to maintain their superior traits and form cloned lines of individual plants of male parents and female parents. The cloned lines of male parents and female parents of selected and matched good combinations were colonized at a ratio of preferably about 1:1 to carry out population hybridization of the two lines, and the F₁ generation hybridized seeds were harvested in a mixed way.

(3) Stabilization of Traits by Asexual Propagation

Individual plants with luxuriant growth, high yield of leaves, strong resistance, similar blooming periods, high seed-setting rate, and high content of total glycoside and RA were selected from the F₁ generations by using the methods in the steps described above, then asexual propagation was carried out to maintain their superior traits.

(4) Superior F₁ generations after their traits are stabilized were used as female parents and the original asexual male parents were used as male parents to carry out backcrossing, the male parents and female parents were colonized at a ratio of preferably about 1:3, and the F₂ generation seeds were harvested.

(5) The harvested F₂ generations were used as female parents and the original asexual male parents were used as male parents to carry out backcrossing, the male parents and the female parents were colonized at a ratio of preferably about 1:3, and the F₃ generation seeds were harvested so as to obtain the target variety.

As compared with other existing varieties the present invention has the following advantages:
1. The characteristics of hybridized F₃ Stevia rebaudiana are: upright branches and stems, lodging resistance, luxuriant growth and large and broad leaves.
2. The superior qualities of the novel hybridized F₃ Stevia rebaudiana variety are: high yield of dry leaves per Chinese acre which is greatly increased relative to that of other varieties; the total content of glycoside and content of rebaudioside A (RA) are also greatly increased relative to those of sexual cultivars; and high resistance, which shows a stronger disease resistance and a stronger pest resistance than other varieties.

The method for systematic breeding of male parents and female parents of cloned lines of Stevia rebaudiana for cultivating a novel elite is based on the following discoveries:
Stevia rebaudiana has the trait of being capable of both sexual propagation and asexual propagation,
using asexual propagation to stabilize superior traits
heterosis; the old Stevia rebaudiana varieties can be replaced with new ones successively and the quality thereof can be improved successively if the breeding goal of “three high and one resistance” is focused on and adhered to successively;
novel combinations can be selected and novel Stevia rebaudiana varieties can be cultivated according to the demands of the market.

Since the cultivation of the novel hybridized varieties of Stevia rebaudiana is low in cost and superior in efficiency, all of the existing varieties of sexual cultivation and asexual cultivation would necessarily be partially or fully replaced by novel hybridized varieties of Stevia rebaudiana and generous economic and social benefits would ensue.

The above-mentioned F₂ generation hybridized seeds which were used as female parents were preferably selected from F₂ generations with superior traits; and their traits were stabilized through asexual propagation and isolated management.

What has been discovered, with the scope of the present invention are:
1) a novel elite variety of Stevia rebaudiana with high yield of TSG in leaves, high content of total steviol glycosides, high content of RA and wherein preferably the Stevia rebaudiana bertonia variety of the present invention comprises greater than 6% by weight RA in the leaf (leaf content), more preferably 7-20% by weight RA in the leaf, and from about 15-28% total steviol content (TSG) in leaf (leaf content), more preferably 14-25% by weight TSG in the leaf, both of which metrics (RA and TSG) are exceptionally high; and wherein of the TSGs content in the leaf, the amount of RA is in a range higher than expected, namely, from 60-85% by weight, more preferably 70-80% by weight and even more preferred 76% by weight;
2) a method for breeding Stevia rebaudiana with a high content of RA, which comprises the following steps: selecting a plant with a RA content in the leaf of greater than 6% by weight (“core plant”), reproducing the core plant to produce parent plants, hybridizing parent plants to produce F₁ generation seeds, and stabilizing the traits of the F₁ generation (namely, in the leaves of the F₁ generation, an RA content of greater than 6% by weight, and (optionally) a TSG of at least 7-20%) and producing F₂ generation seeds by a backcross method; the present invention having the advantages of high yield of leaves, high content of total steviol glycoside, high content of rebaudioside C (RA), strong resistance, and stable traits of plants;
3) a natural sweetener composition comprising RA extracted and purified from any of the plant material as described herein;
4) a natural flavour composition comprising RA extracted and purified from any of the plant material as described herein;
5) a natural sweetener composition comprising a blend of Rebaudioside A extract (produced from the varietal of the present invention) along with at least one or both of Stevioside (STV) extract and Rebaudioside C extract wherein the relative weight percent of Rebaudioside A is higher than in known extracted compositions and said composition optionally comprising at least one secondary sweetener, wherein said secondary sweetener may be Luo Han Guo.
6) a purification process for extracting, from the Stevia rebaudiana elite variety, described herein, a composition comprising a blend of Rebaudioside A extract (produced from the varietal of the present invention) along with one or both of Stevioside (STV) extract and Rebaudioside C extract wherein the relative weight percent of Rebaudioside A is higher than in known extracted compositions; and
7) food, beverage and supplement formulations comprising a natural composition comprising RA extracted and purified from any of the plant material as described herein.

Genetic Identification of the Variety of the Invention

In the means by which to classify a variety based on differences in DNA, a genome DNA is singly isolated from a plant by CTAB, ribonucleic acid (RNA) is removed, and a PCR amplified product obtained by the PCR method by use of a primer mix is distinguished by the differences in DNA finger print obtained by the agarose gel electrophoresis method. In the case of the plant variety in accordance with the present invention, there is provided an identifying band conformation as follows: three specific bands at between i) 500 bp-750 bp, ii) 750-1000 bp; and iii) about 2000 bp, in the DNA marker chart used by CDDP (Conserved DNA-derived Polymorphism) method amplified by the primer of Myb 1: GGCAAGGGCTGCCGC and when analyzed by Random Amplified Polymorphic DNA (RAPD) using the primers of SEQ ID NO: 1, SEQ ID NO: 2, SEQ ID NO: 3 and SEQ ID NO: 4.

Prior to the findings of the present invention, Rebaudioside A, while a component of prior known Steviol Glycoside extracts, was conventionally costly to “selectively” extract and distill from the mother liquor of leaves. The finding of a new varietal of plant, with an expectedly higher amount of Rebaudioside A, is a game changer.

In regards to the leaf extraction process, from this new varietal of plant, with an expectedly higher amount of Rebaudioside A, it can employ aqueous solvents or alcohol/ketone based solvents.

In regards to the formulation, both the sweetening and flavouring agent properties of the “high RA” composition have proven to be excellent.

Extraction and Purification

Typically, steviol glycosides are obtained by extracting leaves of the new Stevia rebaudiana varietal, described herein, with water or alcohols (ethanol or methanol); the obtained extract is a dark particulate solution containing all the active principles plus leaf pigments, soluble polysaccharides, and other impurities. Some processes remove the “grease” from the leaves with solvents such as chloroform or hexane before extraction occurs. There are dozens of extraction patents for the isolation of steviol glycosides, such processes often being categorized by the extraction patents into those based on solvent, solvent plus a decolorizing agent, adsorption and column chromatography, ion exchange resin, and selective precipitation of individual glycosides. Methods using ultrafiltration, metallic ions, supercritical fluid extraction with CO₂ and extract clarification with zeolite are found within the body of more recent patents.

Natural Sweetener Compositions

Natural sweetener compositions that have a taste profile comparable to sugar are desired. Further, a composition that is not prohibitively expensive to produce is preferred. Such a composition can be added, for example, to beverages and food products to satisfy consumers looking for a sweet taste. There is provided herein a process to selectively extract particular steviol glycosides in order to customize sweetening goals

The genus Stevia consists of about 240 species of plants native to South America, Central America, and Mexico, with several species found as far north as Arizona, New Mexico, and Texas. They were first researched by Spanish botanist and physician Petrus Jacobus Stevus (Pedro Jaime Esteve), from whose surname originates the Latinized word stevia.

Steviol glycosides have highly effective sweet taste properties. In fact, these compounds range in sweetness up to 380 times sweeter than sucrose. They are safe, non-toxic heat-stable, pH-stable, and do not ferment making them very commercially workable in the manufacture of foods and beverages. Furthermore, they do not induce a glycemic response when ingested (they have zero calories, zero carbohydrates and a zero glycemic index), making them extremely attractive as natural sweeteners to diabetics, those on carbohydrate-controlled diets and to anyone seeking healthy alternatives. The glycemic index, or GI, measures how fast a food will raise blood glucose level. Choosing foods that produce zero fluctuations in blood glucose is an important component for long-term health and reducing risk of heart disease and diabetes. As such, use of the natural sweetener compositions of the present invention has enormous advantages over cane, beet and other sugars.

Typically, steviol glycosides are obtained by extracting leaves of Stevia rebaudiana Bertoni with hot water or alcohols (ethanol or methanol); the obtained extract is a dark particulate solution containing all the active principles plus leaf pigments, soluble polysaccharides, and other impurities. Some processes remove the “grease” from the leaves with solvents such as chloroform or hexane before extraction occurs. There are dozens of extraction patents for the isolation of steviol glycosides, such processes often being categorized the extraction patents into those based on solvent, solvent plus a decolorizing agent, adsorption and column chromatography, ion exchange resin, and selective precipitation of individual glycosides. Methods using ultrafiltration, metallic ions, supercritical fluid extraction with CO₂ and extract clarification with zeolite are found within the body of more recent patents.

At the 68th Joint Expert Committee on Food Additives (“JECFA”) meeting in 2007, steviol glycosides were defined as the products obtained from the leaves of Stevia rebaudiana Bertoni. As cited by JECFA, the typical manufacture starts with extracting leaves with hot water and the aqueous extract is passed through an adsorption resin to trap and concentrate the component steviol glycosides. The resin is washed with methanol to release the glycosides and the product is recrystallized with methanol. Ion-exchange resins may be used in the purification process. The final product is commonly spray-dried. Table 2 (at the conclusion of the disclosure) provides a product monograph of steviol glycosides, including chemical names, structures, methods of assay and sample chromatogram showing elution times of nine major glycosides.

In the process of purifying high content RA from a stevia leaf extract there are further optional downstream refining steps.

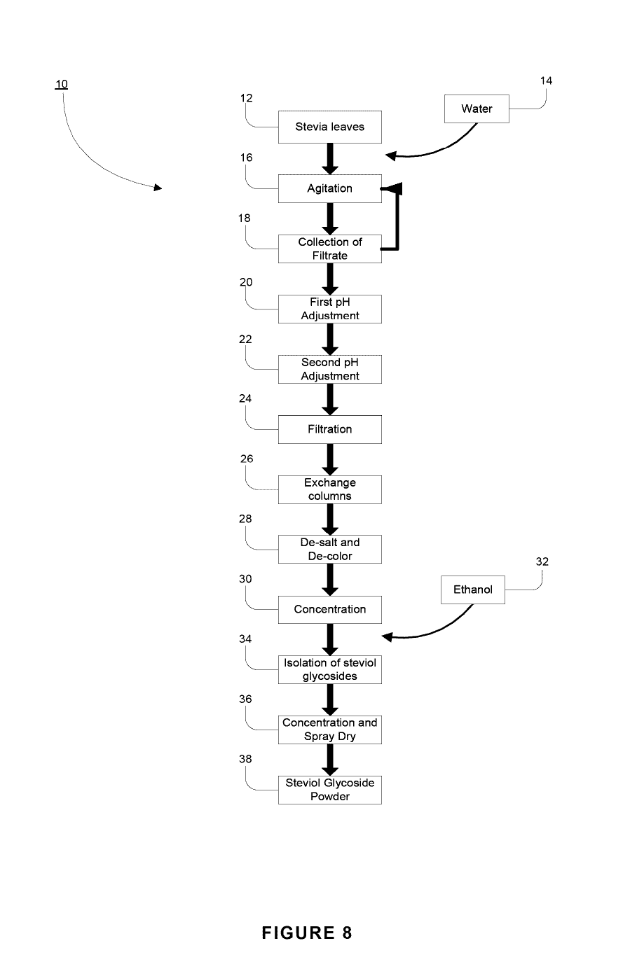

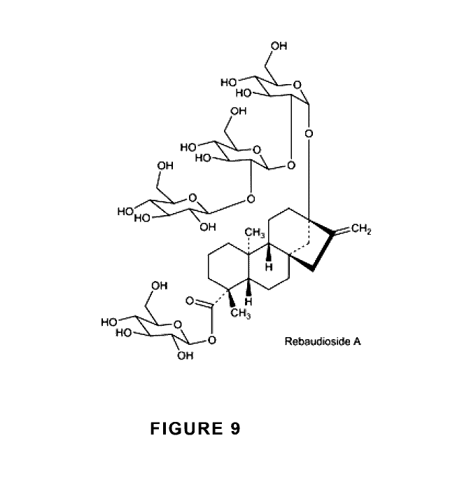

The following provides preferred steps of an extraction process used to isolate glycoside extracts (yielding mother liquor) from Stevia leaves. As shown in FIG. 8, the Stevia leaves (12) are dried and the dried stevia leaves are agitated (16) in a volume of water (14) to release the sweet glycosides from the dried stevia leaves. Preferably, the sweet glycosides are released from the dried leaves using between about 1 volume to about 15 volumes of water. Even more preferably, the sweet glycosides are released from the dried leaves using about 12 volumes of water. The water-leaves mixture is agitated (16) for a period of time between about 10 minutes and about 1 hour, more preferably for a period of time between about 25 minutes and about 35 minutes. Following the agitation (16), the water-leaves mixture is drained and the filtrate collected (18). The cycle of agitation (16) and the collection of filtrate (18) is repeated for a total of about five cycles. Over the course of the five cycles, the water-leaves mixture is agitated for a total period of time between about 1 hour and about 5 hours, more preferably for a total period of time between about 2 hours and about 3 hours.

In one embodiment, for each agitation/collection cycle, the water-leaves mixture is agitated (16) in an environment having a temperature between about 5° C. and about 50° C., more preferably at a temperature between about 20° C. and about 30° C. Following the completion of the agitation/collection cycles, the pH of the water-leaves mixture is first adjusted to about pH 8.0 (20). The pH adjusted water/leaves mixture is then allowed to stand for a period of time between about 30 minutes and about two hours. The pH of the water-leaves mixture is then adjusted a second time (22) to about pH 7.0. The water-leaves mixture is subsequently filtered (24) to obtain an aqueous filtrate. The aqueous filtrate is then applied to ion exchange columns (26) to purify and decontaminate the aqueous filtrate. A person skilled in the art would understand that other methods may also be used to purify and decontaminate the aqueous filtrate. The aqueous filtrate is subsequently de-salted and de-colorized (28) and concentrated (30) using adsorption resin beds. A person skilled in the art would understand that other methods may also be used to concentrate the aqueous filtrate. A filtrate solution containing concentrated steviol glycosides is released from the adsorption resin beds (34) by rinsing the adsorption resin beds with ethanol (32), preferably about 70% ethanol (32).

In a further aspect, the present invention provides a process for producing a natural sweetening composition comprising at least an RA extract, said process comprising the steps of: those detailed in the preceding paragraph, then the stevia primary extract is further processed with additional purification steps to obtain the high purity RA. The stevia primary extract is dissolved in ethanol and/or methanol, crystallized and filtered. The crystallization and drying process is repeated one or several more times using ethanol and/or methanol to obtain high purity RA crystals. The RA content in the final product will reach up to or above 95%, while the total steviol glycosides (TSG) content will reach up to or above 97%. The RA crystals are separated by plate filtration and spray dried to obtain the dry powder product.

In one embodiment, Stevia leaves known to have a high content of RA are used to obtain a RA extract (primary and/or further purified) between about 40% and about 98% purity, optionally from 60-95% purity.

Natural Sweetener Compositions

Natural sweetener compositions that have a taste profile comparable to sugar are desired. Further, a composition that is not prohibitively expensive to produce is preferred. Such a composition can be added, for example, to beverages and food products to satisfy consumers looking for a sweet taste. There is provided herein a process to selectively extract particular steviol glycosides in order to customize sweetening goals.

By way of further background, the genus Stevia consists of about 240 species of plants native to South America, Central America, and Mexico, with several species found as far north as Arizona, New Mexico, and Texas. They were first researched by Spanish botanist and physician Petrus Jacobus Stevus (Pedro Jaime Esteve), from whose surname originates the Latinized word stevia.

Steviol glycosides have highly effective sweet taste properties. In fact, these compounds range in sweetness up to 380 times sweeter than sucrose. They are safe, non-toxic, heat-stable, pH-stable, and do not ferment making them very commercially workable in the manufacture of foods and beverages. Furthermore, they do not induce a glycemic response when ingested (they have zero calories, zero carbohydrates and a zero glycemic index), making them extremely attractive as natural sweeteners to diabetics, those on carbohydrate-controlled diets and to anyone seeking healthy alternatives. The glycemic index, or GI, measures how fast a food will raise blood glucose level. Choosing foods that produce zero fluctuations in blood glucose is an important component for long-term health and reducing risk of heart disease and diabetes. As such, use of the natural sweetener compositions of the present invention has enormous advantages over cane, beet and other sugars.

As noted above, it has been presently found that RA, a glycoside generally present in steviol extracts is found in significantly higher concentrations in extracts of the new plant varietal described herein.

Formulations

A further aspect of the present invention provides a solution to the problem of reduction of sugar intake while not sacrificing sweet taste. This composition has a rounded and refreshing mouth feel and up to 100% less calories. The present invention not only overcomes the disadvantages of high calories and health effects due to excessive intake of white sugar, but also utilizes fully the advantage of Stevia sugar in being purely natural, and having a high sweetness, and good safety and stability; and the compounded sweetener has a better mouth-feel and fresher taste, and is safer and more convenient for use, meeting people's demands for reducing calories in diets.

The “high Reb A” composition of the present invention, for use as a sweetener, preferably comprises Reb A, Reb C, STV and one or more of Reb D and Reb M. Said composition, in one aspect, also comprises a secondary sweetening component as described further below. Said composition, in one aspect, also comprises Luo Han Guo (Mogroside V), as described further below.

In an alternative embodiment, the sweetener compositions of the present invention (comprising one or more glycosides extracted and distilled from the new plant varietal described herein) additionally comprise a secondary sweetening component. The secondary sweetening component is preferably selected from the group consisting of sucrose, erythritol, fructose, glucose, maltose, lactose, corn syrup (preferably high fructose), xylitol, sorbitol, or other sugar alcohols, inulin, miraculin, monetin, thaumatin and combinations thereof, and also non-natural sweeteners such as aspartame, neotame, saccharin, sucralose and combinations thereof. Preferably, for a 50% reduced calorie table top product, the ratio of a secondary sweetening component (most preferably sucrose) to the blends is preferably about 24.7:1. Such a natural sweetener composition can easily be added to food products and beverages, or can be used as a table top sweetener. The ratio of secondary sweetening component to the blends is more preferably between about 5:1 and 1:1. The natural sweetener compositions may be used alone or in combination with other secondary sweeteners, as described herein, and/or with one or more organic and amino acids, flavours and/or coloring agents.

In an alternative embodiment, the sweetener compositions of the present invention (comprising one or more glycosides extracted and distilled from the new plant varietal described herein) additionally comprise an extracted Luo Han Guo mogroside V from the fruit of Grosvenor momordica, more preferably Luo Han Guo mogroside V. Mogroside V is a sweetener free of carbohydrate and calorie, has a sweetness of about 300 times that of sugar, while exhibiting good taste, unique flavor and stable mass. In addition, heating at high temperature, cooking or barbecuing does not affect the flavor of mogroside V, so it can be widely used as an ingredient in foods and beverage.

The sweetener compositions of the present invention (comprising one or more glycosides extracted and distilled from the new plant varietal described herein) may be used in the preparation of various food products, beverages, medicinal formulations, chemical industrial products, among others. Exemplary applications/uses for the sweetener compositions include, but are not limited to: (a) food products, including canned food, preserved fruits, pre-prepared foods, soups, (b) beverages, including coffee, cocoa, juice, carbonated drinks, sour milk beverages, yogurt beverages, meal replacement beverages, and alcoholic drinks, such as brandy, whisky, vodka and wine; (c) grain-based goods—for example, bread and pastas, cookies, pastries, whether these goods are cooked, baked or otherwise processed; (d) fat-based products—such as margarines, spreads (dairy and non-dairy), peanut butter, peanut spreads, and mayonnaise; (d) Confectioneries—such as chocolate, candies, toffee, chewing gum, desserts, non-dairy toppings (for example Cool Whip®), sorbets, dairy and non-dairy shakes, icings and other fillings, (e) drug and medicinal formulations, particularly in coatings and flavourings; (f) cosmetics and health applications, such as for sweetening toothpaste; and (g) seasonings for various food products, such as soy sauce, soy sauce powder, soy paste, soy paste powder, catsup, marinade, steak sauce, dressings, mayonnaise, vinegar, powdered vinegar, frozen-desserts, meat products, fish-meat products, potato salad, bottled and canned foods, fruit and vegetables.

The natural sweetener compositions of the present invention may be formulated into premixes and sachets. Such premixes may then be added to a wide variety of foods, beverages and nutraceuticals. The purified natural sweetener compositions may, in one preferred form, be table top sweeteners.

The present invention provides a method for breeding Stevia rebaudiana with a high content of RA, which comprises the following steps: selecting a plant with a RA content in the leaf of greater than 6% by weight (“core plant”), asexually reproducing the core plant to produce parent plants, hybridizing the parent plants to produce F₁ generation seeds, and stabilizing the traits of the F₁ generation (namely, in the leaves of the F₁ generation, producing an RA content of at least 7-20% (preferably around 16%) by weight, and a TSG of at least 15-28% (preferably around 21%) and thereafter producing F₂ generation seeds by a backcross method.

The present invention further provides a method for breeding Stevia rebaudiana with a high content of RA, which comprises the following steps:
(1) selecting a plant with a RA content in the leaf of greater than 6% (“core plant”);
(2) asexually reproducing the core plant to produce parent plants;
(3) hybridizing parent plants to produce F₁ generation seeds;
(4) stabilizing the traits of the F₁ generation (namely, in the leaves of the F₁ generation, an RA content of at least 7-20% by weight, and a TSG of at least 15-28%);
(3) producing F₂ generation seeds by a backcross method; and
(4) producing F₃ generation seeds by a backcross method.

The present invention further provides Stevia rebaudiana elite variety seeds, cells, plants, germplasm, breeding lines, varieties, and plant parts produced by these methods.

The present invention further provides foods, beverages, nutraceuticals, functional foods, medicinal formulations, cosmetics, health products, condiments and seasonings comprising steviol glycoside compositions extracted and purified from leaf plant material of the new varietal described and claimed herein.

The present invention further provides a steviol glycoside compositions additionally comprising at least one secondary sweetener. The present invention further provides a steviol glycoside compositions additionally comprising Luo Han Guo (Mogroside V).

While the forms of processes and compositions described herein constitute preferred embodiments of this invention, it is to be understood that the invention is not limited to these precise forms. As will be apparent to those skilled in the art, the various embodiments described above can be combined to provide further embodiments. Aspects of the present composition, method and process (including specific components thereof) can be modified, if necessary, to best employ the systems, methods, nodes and components and concepts of the invention. These aspects are considered fully within the scope of the invention as claimed. For example, the various methods described above may omit some acts, include other acts, and/or execute acts in a different order than set out in the illustrated embodiments.

Further, in the methods taught herein, the various acts may be performed in a different order than that illustrated and described. These and other changes can be made to the present systems, methods and articles in light of the above description. In general, in the following claims, the terms used should not be construed to limit the invention to the specific embodiments disclosed in the specification and the claims, but should be construed to include all possible embodiments along with the full scope of equivalents to which such claims are entitled. Accordingly, the invention is not limited by the disclosure, but instead its scope is to be determined entirely by the following claims.

All publications, patents and patent applications mentioned in the specification are indicative of the level of those skilled in the art to which this invention pertains. All such publications, patents and patent applications are incorporated by reference herein for the purpose cited to the same extent as if each was specifically and individually indicated to be incorporated by reference herein.

The sweetener compositions of the present invention (comprising one or more glycosides prepared by the processes described herein) may be used in the preparation of various food products, beverages, medicinal formulations, chemical industrial products, among others. Exemplary applications/uses for the sweetener compositions include, but are not limited to: (a) food products, including canned food, preserved fruits, pre-prepared foods, soups, (b) beverages, including coffee, cocoa, juice, carbonated drinks, sour milk beverages, yogurt beverages, meal replacement beverages, and alcoholic drinks, such as brandy, whisky, vodka and wine; (c) grain-based goods—for example, bread and pastas, cookies, pastries, whether these goods are cooked, baked or otherwise processed; (d) fat-based products—such as margarines, spreads (dairy and non-dairy), peanut butter, peanut spreads, and mayonnaise; (d) Confectioneries—such as chocolate, candies, toffee, chewing gum, desserts, non-dairy toppings (for example Cool Whip®), sorbets, dairy and non-dairy shakes, icings and other fillings, (e) drug and medicinal formulations, particularly in coatings and flavourings; (f) cosmetics and health applications, such as for sweetening toothpaste; and (g) seasonings for various food products, such as soy sauce, soy sauce powder, soy paste, soy paste powder, catsup, marinade, steak sauce, dressings, mayonnaise, vinegar, powdered vinegar, frozen-desserts, meat products, fish-meat products, potato salad, bottled and canned foods, fruit and vegetables.

The natural sweetener compositions of the present invention may be formulated into premixes and sachets. Such premixes may then be added to a wide variety of foods, beverages and nutraceuticals. The purified natural sweetener compositions may, in one preferred form, be table top sweeteners.

In an alternative embodiment, the sweetener compositions of the present invention (comprising one or more glycosides prepared by the processes described herein) additionally comprise a secondary sweetening component. The secondary sweetening component is preferably selected from the group consisting of sucrose, erythritol, fructose, glucose, maltose, lactose, corn syrup (preferably high fructose), xylitol, sorbitol, or other sugar alcohols, inulin, miraculin, monetin, thaumatin and combinations thereof, and also non-natural sweeteners such as aspartame, neotame, saccharin, sucralose and combinations thereof. Preferably, for a 50% reduced calorie table top product, the ratio of a secondary sweetening component (most preferably sucrose) to the blends is preferably about 24.7:1. Such a natural sweetener composition can easily be added to food products and beverages, or can be used as a table top sweetener. The ratio of secondary sweetening component to the blends is more preferably between about 5:1 and 1:1. The natural sweetener compositions may be used alone or in combination with other secondary sweeteners, as described herein, and/or with one or more organic and amino acids, flavours and/or coloring agents.

While the forms of processes and compositions described herein constitute preferred embodiments of this invention, it is to be understood that the invention is not limited to these precise forms. As will be apparent to those skilled in the art, the various embodiments described above can be combined to provide further embodiments. Aspects of the present composition, method and process (including specific components thereof) can be modified, if necessary, to best employ the systems, methods, nodes and components and concepts of the invention. These aspects are considered fully within the scope of the invention as claimed. For example, the various methods described above may omit some acts, include other acts, and/or execute acts in a different order than set out in the illustrated embodiments.

Further, in the methods taught herein, the various acts may be performed in a different order than that illustrated and described. Additionally, the methods can omit some acts, and/or employ additional acts.

These and other changes can be made to the present systems, methods and articles in light of the above description. In general, in the following claims, the terms used should not be construed to limit the invention to the specific embodiments disclosed in the specification and the claims, but should be construed to include all possible embodiments along with the full scope of equivalents to which such claims are entitled. Accordingly, the invention is not limited by the disclosure, but instead its scope is to be determined entirely by the following claims.

All publications, patents and patent applications mentioned in the specification are indicative of the level of those skilled in the art to which this invention pertains. All such publications, patents and patent applications are incorporated by reference herein for the purpose cited to the same extent as if each was specifically and individually indicated to be incorporated by reference herein.

The following examples illustrate preferred embodiments of the present invention.

### EXAMPLES

### Example 1

### Breeding

Select a plant (from 80-95 cm in height) with a RA content in the dried leaf of greater than 6% by weight (“core plant”). Reproduce the core plant to produce male and female parent plants. Asexually reproduce the parent plants. Hybridize to produce F₁ generation seeds, and stabilize the traits of the F₁ generation (namely, in the leaves of the F₁ generation, an RA content of at least 7-20% by weight and producing F₂ generation seeds by a backcross method

### Example 2

### Breed Testing-Genetic Identification

To identify the steviol glycosides of the new varietal including component ratio and yield, as the Morita variety was the best, the Morita variety was identified as a standard by the PCR method.

A mortar sterilized by drying and heating was charged with about 0.2 g of the leaves of the Morita variety, to which liquid nitrogen was added, they were crushed by a pestle, and about 0.05 g at a time was put into micro tube(s) by a spatula. 300 μl of 2% CTAB solution (2% CTAB solution (50 ml): composition 100 mM, tris-HCl (pH 8.0) 20 mM, EDTA (pH 8.0), 2% CTAB, 1.4 M NaCl) was added thereto, and after tumbling and mixing, the tube was moved to a heat block heated to 65° C., and it was heated for 30 min. An equal amount (300 μl) of chloroform/isoamyl alcohol (24:1) was added thereto, followed by stirring gradually. After centrifuging and separating it at 14000 rpm for 15 min, the aqueous layer, which was the upper layer of the content separated into 2 layers, was moved to a new tube.

The operations after above-mentioned chloroform/isoamyl alcohol were repeated one more time, and an aqueous layer was moved to a new tube. 400 μl of 1% CTAB solution (1% CTAB solution (50 ml): composition 1 M, tris-HCl 2.5 mM, EDTA 1.0 ml, 1% CTAB 0.5 g) was added thereto, and after tumbling and mixing for 15 min, it was left to stand still at room temperature for 1 hr, followed by centrifugal separation at 14000 rpm for 15 min.

The supernatant was discarded, followed by the addition of 400 μl of 1 MCsCl, and the precipitate was completely dissolved by pipetting. 900 μl of 100% ethanol was added thereto, and after tumbling and mixing, it was left to stand still at a temperature of −20° C. for 20 min, followed by centrifugal separation at 14000 rpm for 15 min. The supernatant was discarded, followed by the addition of 400 μl of 70% ethanol to the precipitate, it was subjected to centrifugal separation at 14000 rpm for 15 min, and after repeating this operation, the supernatant was discarded, the precipitate was dried in a vacuum dryer, and it was dissolved in 30 μl of extra pure water. The solution was subjected to the agarose gel electrophoresis, thereby confirming that DNA was separated alone.

In order to remove RNA, it was allowed to undergo a reaction in 500 μl of an RNase solution (composition: 100 μl of the above-mentioned DNA isolated solution and 5 μl of RNase (5 g/ml) at 37° C. for 1 hr, and an equal amount of the PCI solution (composition: a solution obtained by centrifuge at 13000 rpm for 5 min and by separating an aqueous layer after mixing phenol/chloroform/isoamyl alcohol (25:24:1) gradually) was added to the reaction solution. After putting a lid and gradually mixing, it was centrifuged at 13000 rpm for 5 min.

The aqueous layer (upper layer) was transferred to a new micro tube, to which an equal amount of the CIA solution (composition: chloroform/isoamyl alcohol, ratio by volume 24:1) preserved at room temperature was added, and after gradually mixing, it was centrifuged at 15000 rpm for 3 min, the aqueous layer was transferred to a new micro tube, followed by the CIA treatment one more time, 3 M sodium acetate of a quantity of 1/10 time that of the supernatant obtained and 100% ethanol of 2.5 times in quantity were added thereto, followed by mixing well, and cooling at −20° C. for 20 min or longer, and then it was centrifuged at 15000 rpm for 15 min, thereby pelletizing DNA, the supernatant was discarded, and after adding to the pellets 1 ml of 70% ethanol which had been cooled down, it was centrifuged at 15000 rpm for 15 min, the supernatant was discarded, and after adding 1 ml of 70% ethanol which had been cooled down, it was centrifuged at 15000 rpm for 15 min, the supernatant was discarded, and it was dried for 5 min by use of a desiccator under a reduced pressure.

All leaf samples were such prepared, using this methodology.

With the genome DNA thus obtained as a template, using PCR composition, a 35 cycle reaction was carried out at 94° C. (30 seq), 55° C. (30 sec), and 72° C. (120 sec), and thereafter, it was allowed to undergo a reaction at 72° C. for 10 min. After the reaction, it was kept at 4° C. and a PCR amplified product was obtained. When the DNA band in the PCR amplified product was confirmed by the 1% agarose gel electrophoresis, a characteristic DNA fragment was confirmed: noting that amplification bands are scarce, and they mostly concentrated in size between i) 500 bp-750 bp, ii) 750-1000 bp; and iii) about 2000 bp, when analyzed by Random Amplified Polymorphic DNA (RAPD) using the primers of SEQ ID NO: 1, SEQ ID NO: 2, SEQ ID NO: 3 and SEQ ID NO: 4.

Preferred Method Conditions and Results:

The CDDP (Conserved DNA-derived Polymorphism) method used for the identification in the present invention is a molecular analytical method of DNA, and it is a method for the analysis by electrophoresis of a DNA pattern amplified in a DNA conserved region, using a single primer in a PCR reaction (Polymerase chain reaction).

In addition, for cetyl trimethyl ammonium bromide (CTAB) is a quaternary ammonium salt having a long chain alkyl group, and it forms an insoluble complex with a poly anion such as nucleic acid, it can be utilized for isolating a nucleic acid. In the means by which to classify a variety based on differences in DNA, the genome DNA is singly isolated from a plant by CTAB, ribonucleic acid (RNA) is removed, a PCR amplified product obtained by the PCR method. CDDP amplification reaction system: The total system of PCR is 20 μL. The genome DNA obtained as a template, Including 2×Es Taq Master Mix (10 μL), Primer (10 μM, 1 μL), Template DNA (2 μL), with ddH2O supplement to 20 μL, undergoing PCR reaction (using the primer Myb1: GGCAAGGGCTGCCGC): after 94° C. 5 min high-temperature pre-denaturation, a 35 cycle reaction was carried out at 94° C. (1 min), 50° C. (1 min), and 72° C. (2 min), and thereafter, it was allowed to undergo a extending reaction at 72° C. for 10 min. After the reaction, it was kept at 4° C. At last, the PCR amplified product was confirmed by the agarose gel electrophoresis method, and thus the plant could be confirmed by a specific DNA band.

Identification Result:

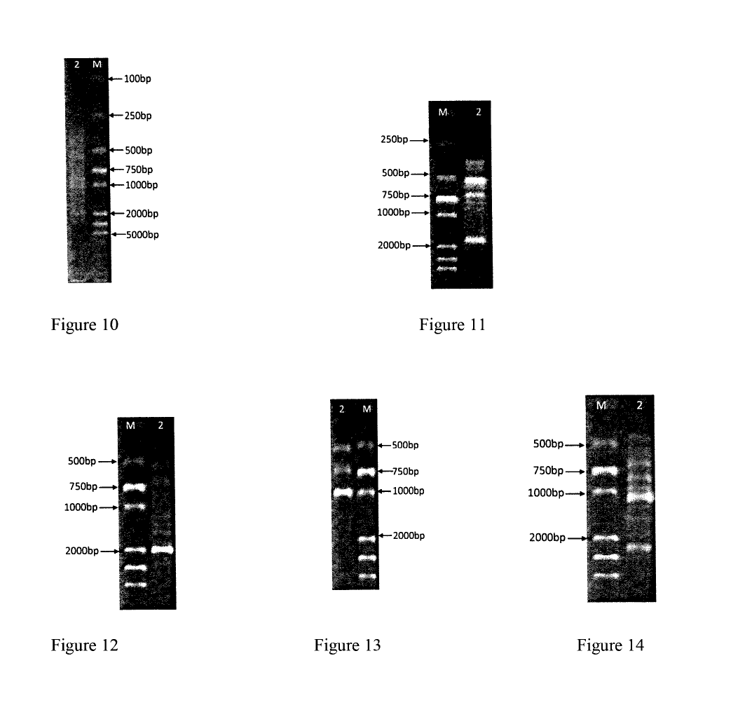

As seen in FIGS. 10-14, Sample 1 (high RA variety of the invention) of stevia amplification bands are scarce, and they mostly concentrated in size between i) 500 bp-750 bp, ii) 750-1000 bp; and iii) about 2000 bp, when analyzed by Random Amplified Polymorphic DNA (RAPD) using the primers of SEQ ID NO: 1, SEQ ID NO: 2, SEQ ID NO: 3 and SEQ ID NO: 4 using CDDP (Conserved DNA-derived Polymorphism) method amplified by the primer of Myb 1: GGCAAGGGCTGCCGC.

In FIG. 10, 1=GLG-High-RA cultivar M=DM 2000 plus Marker.

Four DNA fingerprinting maps using four primers were employed:

(FIG. 11) SEQ ID NO: 1 (Primer 2):
5′-TTTGGTGACGGTGCGG-3′
(FIG. 12) SEQ ID NO: 2 (Primer 3):
5′-TGGGGCCAACCCAAGTC-3′
(FIG. 13) SEQ ID NO: 3 (Primer 4):
5′-GGCCTGCAGCTCTTCT-3′
(FIG. 14) SEQ ID NO: 4 (Primer 5):
5′-GCGTCCCCAACTCGATC-3′

### Example 3

### Plant Materials

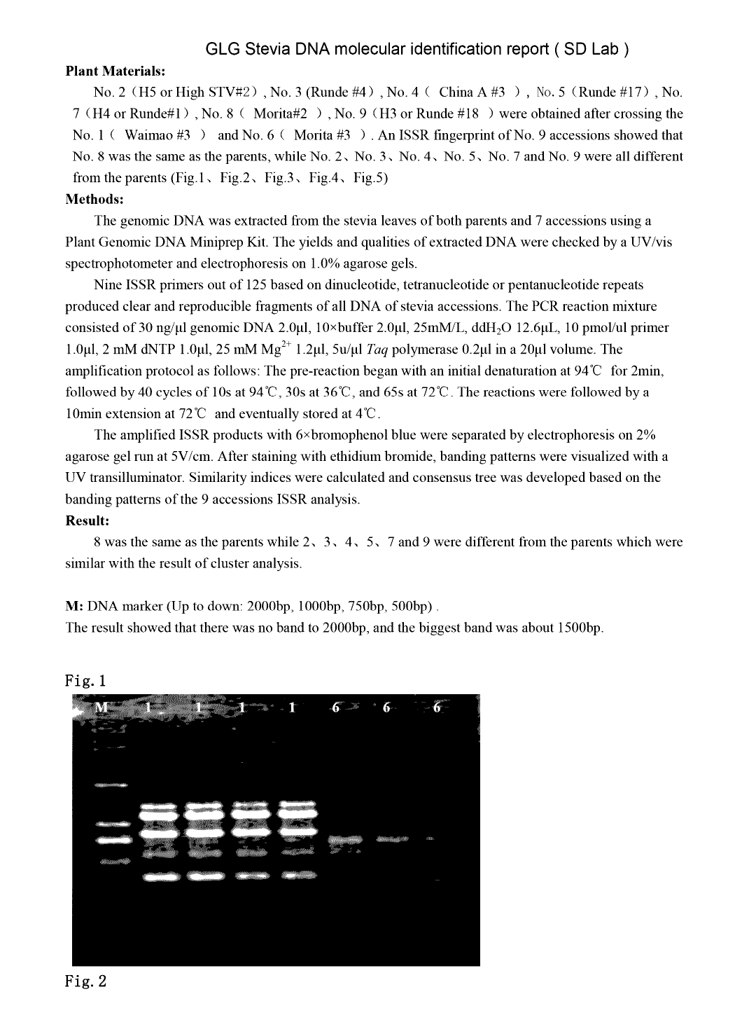

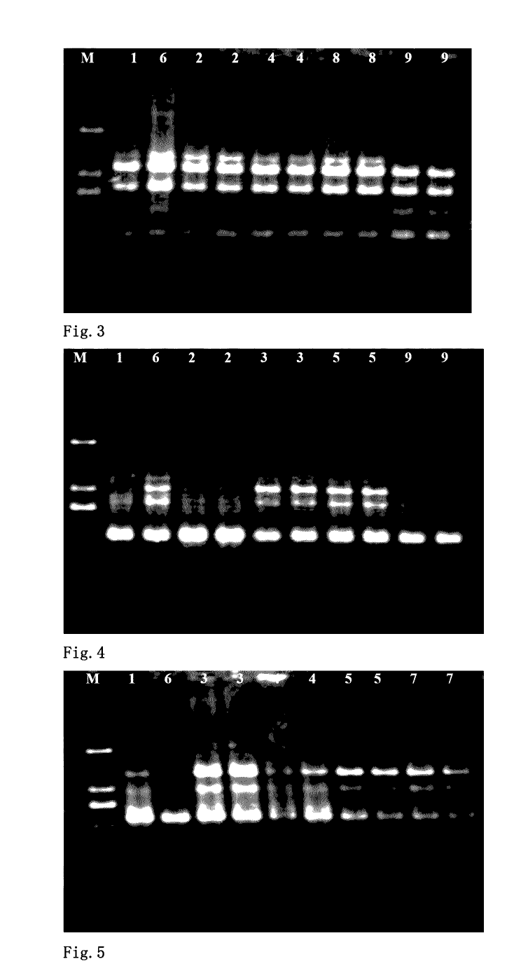

No. 2 (H5 or High STV #2), No. 3 (Runde #4), No. 4 (China A #3), No. 5 (Runde #, No. 7 (H4 or Runde #1, No. 8 (Morita #2, No. 9 (H3 or Runde #18) were obtained after crossing the No. 1 (Waimao #3) and No. 6 (Morita #3. An ISSR fingerprint of No. 9 accessions showed that No. 8 was the same as the parents, while No. 2, No. 3, No. 4, No. 5, No. 7 and No. 9 were all different from the parents (refer to FIGS. 1-5)

Methods:

The genomic DNA was extracted from the stevia leaves of both parents and seven accessions using a Plant Genomic DNA Miniprep Kit. The yields and qualities of extracted DNA were checked by a UV/vis spectrophotometer and electrophoresis on 1.0% agarose gels.

Nine ISSR primers out of 125 based on dinucleotide, tetranucleotide or pentanucleotide repeats produced clear and reproducible fragments of all DNA of stevia accessions. The PCR reaction mixture consisted of 30 ng/μl genomic DNA 2.0 μl, 10× buffer 2.0 μl, 25 mM/L, ddH₂O 12.6 μL, 10 pmol/ul primer 1.0 μl, 2 mM dNTP 1.0 μl, 25 mM Mg²⁺1.2 μl, 5 u/μl Taq polymerase 0.2 μl in a 20 μl volume.

The amplification protocol was as follows: the pre-reaction began with an initial denaturation at 94□ for 2 min, followed by 40 cycles of 10 s at 94□, 30 s at 36□, and 65 s at 72□. The reactions were followed by a 10 min extension at 72□ and eventually stored at 40. The amplified ISSR products with 6× bromophenol blue were separated by electrophoresis on 2% agarose gel run at 5V/cm. After staining with ethidium bromide, banding patterns were visualized with a UV transilluminator. Similarity indices were calculated and consensus tree was developed based on the banding patterns of the 9 accessions ISSR analysis.

Results:

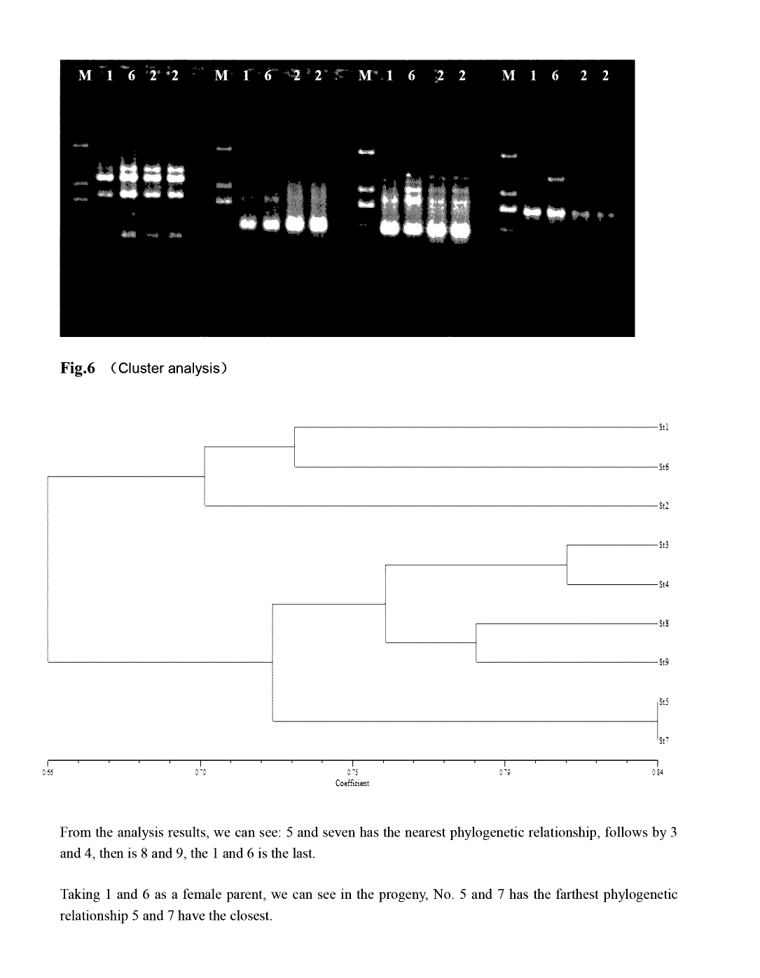

8 was the same as the parents while 2, 3, 4, 5, 7 and 9 were different from the parents which were similar with the result of cluster analysis (FIG. 6).

M: DNA marker (Up to down: 2000 bp, 1000 bp, 750 bp, 500 bp).

The result showed that there was no band to 2000 bp, and the biggest band was about 1500 bp.

From the analysis results, it can be determined that: 5 and 7 have the nearest phylogenetic relationship, followed by 3 and 4, then 8 and 9, with 1 and 6 being the last.

Taking 1 and 6 as a female parent, it can be seen in the progeny, No. 5 and 7 has the farthest phylogenetic relationship 5 and 7 have the closest.

### Example 4

### GLG Stevia H2, H3, H4 and H5 DNA Molecular Identification Report (AT Lab)

Instructions:

A: as Morita #2;

B: as H3 mother (B6);

C: as H5 father;

D: as Morita #3;

E: as H4;

F: as H5;

G: as H3 mother (B4);

H: as H3 mother (J3);

I: as H2;

J: as H3;

K: as DC#1;

L: as C1.

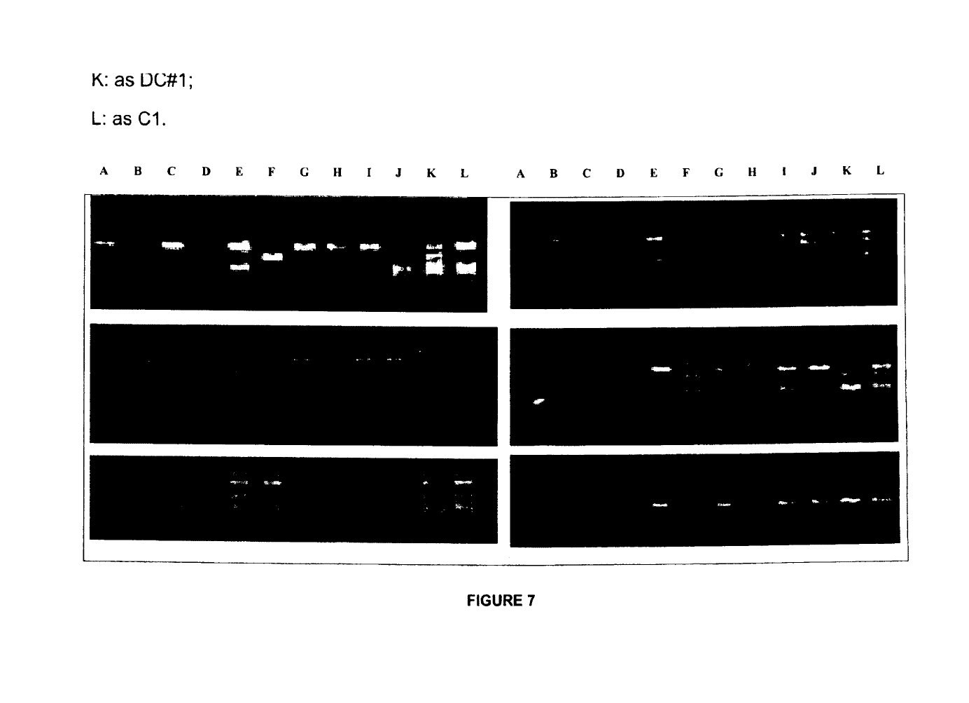

Results: FIG. 7.

### Example 3

### Extraction of Steviol Glycosides from Stevia rebaudiana Leaves

One kg of the stevia leaves known to have a high content of Rebaudioside A were steeped with 2 kg of room temperature water having a pH of 7.3 in an agitation centrifuge. The leaves were agitated for 0.5 hour. The sweet water was filtered, the filtrate collected and the process repeated for a total of 5 steep/separation cycles. The pH of the sweet water filtrate solution was adjusted to pH 8.0 with approximately 30 grams of calcium hydroxide. After a rest time of about 1 hour, 50 grams of FeCl₃ was added to the sweet water filtrate solution to further adjust the pH to 7.0. The solution was filtered and the resulting filtrate had a transmittance of about 68±2% at 325 nm. The filtrate flows through the resin bed, and the glycosides was eluted from the resin bed by using 75% of ethanol. The eluate was concentrated to 45-50% of solid content, and then was vacuum dried. This dried eluate is called stevia extract or Stevia Primary Extract (SPE).

We claim:

1. A Stevia rebaudiana plant that comprises a Rebaudioside A leaf content selected from:
i) greater than 6% by dry weight;
ii) greater than 10% by dry weight;
iii) greater than 15% by dry weight; and
iv) around 16% by dry weight;
and a total steviol glycosides leaf content selected from the group consisting of:
i) 15-28% by dry weight;
ii) 14-25% by dry weight;
iii) 18-23% by dry weight; and
iv) around 21% by dry weight;
and which comprises three bands, one between each of i) 500 bp-750 bp; ii) 750-1000 bp; and iii) about 2000 bp, when analyzed by Random Amplified Polymorphic DNA (RAPD) using the primers of SEQ ID NO: 1, SEQ ID NO: 2, SEQ ID NO: 3 and SEQ ID NO: 4.

2. A Stevia rebaudiana plant that comprises an Rebaudioside A leaf content selected from the group consisting of:
i) greater than 6% by dry weight;
ii) greater than 10% by dry weight;
iii) greater than 15% by dry weight; and
iv) around 16% by dry weight;
and a total steviol glycosides leaf content selected from the group consisting of:
i) 15-28% by dry weight;
ii) 14-25% by dry weight;
iii) 18-23% by dry weight;
iv) around 21% by dry weight;
and a Rebaudioside A content as a percentage of total steviol glycosides selected from the group consisting of:
i) 60-85% by dry weight;
ii) 70-80% by dry weight;
iii) around 76% by dry weight;
and which comprises three bands, one between each of i) 500 bp-750 bp; ii) 750-1000 bp; and iii) about 2000 bp, when analyzed by Random Amplified Polymorphic DNA (RAPD) using the primers of SEQ ID NO: 1, SEQ ID NO: 2, SEQ ID NO: 3 and SEQ ID NO: 4.

2. The Stevia rebaudiana plant of claim 1, that is the Stevia rebaudiana bertoni variety.

3. A method for breeding Stevia rebaudiana with a high content of RA, which comprises the following steps: selecting a plant with a RA content in the leaf of greater than 6% by weight (“core plant”), asexually reproducing the core plant to produce parent plants, hybridizing the parent plants to produce F₁ generation seeds, and stabilizing the traits of the F₁ generation (namely, in the leaves of the F₁ generation, producing an RA content of at least 7-20% by weight and thereafter producing F₂ generation seeds by a backcross method.

4. A method for breeding Stevia rebaudiana with a high content of RA, which comprises the following steps:
(1) selecting a plant with a RA content in the leaf of greater than 6% (“core plant”);
(2) asexually reproducing the core plant to produce parent plants;
(3) hybridizing parent plants to produce F₁ generation seeds;
(4) stabilizing the traits of the F₁ generation (namely, in the leaves of the F₁ generation, an RA content of at least 7-20% by weight;
(3) producing F₂ generation seeds by a backcross method; and
(4) producing F₃ generation seeds by a backcross method.

5. The method of claim 4 wherein parent plants are a male plant and a female plant, and wherein the male parents and female parents which are selected and matched in said step (3) by asexual propagation wherein the clonal plants are colonized at a ratio of 1:1.

6. The method of claim 4 wherein parent plants are a male plant and a female plant and wherein the male plant and the female plant in said step of producing F₂ generation seeds by a backcross method are colonized at a ratio of 1:3.

7. Stevia rebaudiana elite variety seeds, cells, plants, tissue culture, germplasm, breeding lines, varieties, and plant parts produced by the methods of claim 1.

8. Stevia rebaudiana elite variety seeds, cells, plants, tissue culture, germplasm, breeding lines, varieties, and plant parts produced by the methods of claim 2.

9. A method for the production of a sweetener composition characterized in that the sweetener composition comprises RA and is produced from dried leaves of the plant described in claim 1 and wherein RA is extracted with a solvent comprising water.

10. A method for the production of a sweetener composition characterized in that the sweetener composition comprises RA and is produced from dried leaves of the plant described in claim 2 and wherein RA is extracted with a solvent comprising water.

11. A natural sweetener composition comprising a composition comprising RA, extracted and purified from any of the plant material of claim 1.

12. A natural sweetener composition comprising a composition comprising RA, extracted and purified from any of the plant material of claim 2.

13. Foods, beverages, nutraceuticals, functional foods, medicinal formulations, cosmetics, health products, condiments and seasonings comprising a composition comprising RA, extracted and purified from any of the plant material of claim 1.

14. Foods, beverages, nutraceuticals, functional foods, medicinal formulations, cosmetics, health products, condiments and seasonings comprising a composition comprising RA, extracted and purified from any of the plant material of claim 2.

15. A natural sweetener composition comprising a composition comprising RA, extracted and purified from any of the plant material of claim 1 additionally comprising at least one secondary sweetener.

16. A natural sweetener composition comprising a composition comprising RA, extracted and purified from any of the plant material of claim 2 additionally comprising at least one secondary sweetener.

17. A natural sweetener composition comprising a composition comprising RA, extracted and purified from any of the plant material of claim 1 additionally comprising Luo Han Guo (Mogroside V).

18. A natural sweetener composition comprising a composition comprising RA, extracted and purified from any of the plant material of claim 2 additionally comprising Luo Han Guo (Mogroside V).
